(19) United States
(12) Patent Application Publication
(10) Pub. No.: US 2013/0220332 A1
(43) Pub. Date: Aug. 29, 2013

(54) LARYNGEAL MASK WITH ENHANCED INSERTION

(71) Applicants: Kanag Baska, Strathfield (AU); Meenakshi Baska, Strathfield (AU)

(72) Inventors: Kanag Baska, Strathfield (AU); Meenakshi Baska, Strathfield (AU)

(21) Appl. No.: 13/818,511

(22) PCT Filed: Aug. 23, 2011
(86) PCT No.: PCT/AU11/01085

§ 371 Date: May 3, 2013

(30) Foreign Application Priority Data
Aug. 23, 2010 (AU) 2010903758

(51) Int. Cl. A61M 16/04

(52) U.S. Cl. CPC A61M 16/0434
USPC 128/207.15

(57) ABSTRACT

A laryngeal mask for establishing an artificial airway in a patient includes a mask portion having a resilient conformable peripheral portion shaped such that the mask forms a seal with the larynx when the mask is positioned in the laryngopharynx, and an airway tube connected to or formed with the mask for passing gas to the larynx when the mask is properly inserted into the laryngopharynx. The mask portion includes at least one region of reduced wall thickness or enhanced flexibility, and at least one region of increased wall thickness (relative to the reduced wall thickness) or enhanced stiffness (relative to the region of enhanced flexibility), the at least one region of increased wall thickness or enhanced stiffness having a larger longitudinal extent than a longitudinal extent of the at least one region of reduced wall thickness or enhanced flexibility. The resilient conformable peripheral portion may be an inflatable cuff.

### FIELD OF THE INVENTION

The present invention relates to a laryngeal mask suitable for use in establishing an artificial airway in a patient.

### BACKGROUND TO THE INVENTION

Maintenance of a viable airway is critical to patient safety during surgical procedures conducted under general anaesthetic. Maintenance of a viable airway during such surgical procedures had, for many years, been achieved by insertion of an endo-tracheal tube into the patient. The endo-tracheal tube was typically inserted through the oral cavity or nasal cavity, into the larynx, through the vocal cords and into the trachea. As the endo-tracheal tube had to be inserted through the vocal cords, difficulty was often experienced in correctly positioning the endo-tracheal tube. British patent no. 2,111,394 (which corresponds to U.S. Pat. No. 4,509,514) describes a device for maintaining an airway in a patient. The device is described as being an artificial airway device. The device comprises a curved, flexible tube opening at one end into the interior of a hollow mask portion shaped to conform to fit readily into the actual and potential space behind the larynx and to seal around the circumference of the laryngeal inlet without penetrating into the interior of the larynx. Commercial forms of this device have an inflatable collar extending around the periphery of the mask. The inflatable collar is adapted to form the seal around the laryngeal inlet when the collar is inflated. Additionally, the mask portion included an inflatable posterior part which is adapted to press against the back of the throat and thereby increase the sealing pressure around the laryngeal inlet.

British patent no. 2,111,394 states that the shape and (when fitted) the inflatable part or parts of the mask ensure that it approximates closely to the shape of the space between the laryngeal inlet and the walls of the lower part of the throat behind it. Since the walls of tissue forming the back of the throat are relatively rigid, inflation of the mask forces it more tightly against the tissues surrounding the laryngeal inlet, so forming an airtight seal, while tending to anchor the mask in position.

In use of the device described in GB 2,111,394, the device is inserted through the mouth of the patient and down the throat past the epiglottis until the mask comes to rest with its distal end in the base of the throat, lying against the upper end of the normally closed oesophagus. The inflatable ring on the mask is then inflated to seal around the inlet to the larynx. The patient's airway is thus secure and unobstructed and the laryngeal mask can be connected directly to conventional anaesthetic circuit hosing for either positive pressure or spontaneous breathing.

The device of GB 2,111,394 has found wide acceptance in use within the medical community. However, the device of GB 2,111,394 can be difficult to insert into the patient. In particular, insertion of a laryngeal mask into a patient requires that the mask takes a complex course. Difficulties in insertion of laryngeal masks particularly arise at two different portions of the airway passage. The first of these is the palatopharyngeal curve, when the mask must and around to follow that curve. The second difficulty is met in trying to pass the mask behind the larynx.

Insertion of laryngeal masks typically involves the anaesthetist pushing the mask into the patient's airway by gripping the airway tube of the laryngeal mask and pushing downwardly on the airway tube. Therefore, many available laryngeal masks have a quite rigid airway tube to facilitate pushing and insertion by the anaesthetist. There are several laryngeal masks that are commercially available that have quite flexible airway tubes. However, these laryngeal masks typically need an introducer, such as a curved stainless steel guide or introducer, to properly introduce those masks into a patient.

Other airway masks have curved airway tubes to facilitate passage past the palatopharyngeal curve. However, some of these masks can get caught in the larynx, causing the mask to fold over itself, causing airway obstruction and difficulty in airway maintenance. As the airway tube of these masks is curved and fixed, it does not allow the distal end of the mask to slide against the cervical vertebra during insertion and thus the distal end of the mask tends to get caught in the larynx and fold over.

There have been several efforts to design laryngeal masks that attempt to facilitate insertion into the patient's airway. International patent publication number WO 02/32490 describes a non-inflatable artificial airway device made from a resilient material for use as a combined obturator and airway device without penetration into the larynx. The device comprises a preformed flexible saccular chamber having a semirigid hollow stem having an airway tube projecting from a position at or near one end of the chamber at an obtuse angle. The airway tube is able to be flexed perpendicularly up to 90° from the longitudinal axis of the chamber. In particular, the relative stiffness of the stem (airway tube) compared with the relative stiffness of the chamber may cause the chamber to buckle at the foot of the stem when the stem is turned towards the perpendicular. In order to improve the flexing where the stem meets the chamber, the cross-section of the stem is reduced in the vicinity of the chamber. However, this patent states that for quite easy insertion of the device, it may be necessary to use an introducer (a stiff curved rod) with its saccular chamber fitted into the introducer from the first end as far as the toe, so that possible folding of the device is avoided.

United States patent application publication number 2006/0201516 A1 describes an inflatable laryngeal mask that has an integrally formed mask and curved airway portion. The airway tube may include reinforcing ribs in the curved portion to increase rigidity of that part of the tube. These ribs have a wall thickness T5 that exceeds the general wall thickness T4 of the airway tube. In other embodiments, a portion of the airway tube may be provided with reinforcing ribs. Although not referred to in the text of this document, some of the figures of this published US patent application appear to show a reinforcing rib located on the inner side of the curved portion of the airway tube. The function of this rib is not explained.

Another problems that can arise when using an laryngeal masks to establish an artificial airway in patients relates to respiratory obstruction that can arise due to mal positioning of the cuff of the mask portion of the laryngeal mask. This can happen when the head of the patient is rotated towards one side.

It will be understood that the inner diameter of the airway of the laryngeal mask needs to be of sufficient diameter to reduce or minimise the resistance to airflow through it. Therefore, it is important that an airway does not kink at any point at any time during its use. Once a laryngeal mask is inserted into a patient, most of the parts of the device will not be visible. Consequently when an obstruction to the airway occurs during its routine use, the problem can becomes so acute that the Anaesthetist may have no choice than to remove the device and change over to an alternate method to continue the anaesthetic. It will not be possible for an Anaesthetist to pin point the cause and the exact location of the obstruction in the device. More often the exact cause of obstruction becomes a′ wild guess'.

If the head of the patient has to be turned to one side or the other, the mask part of the device may get rotated to an abnormal position causing respiratory obstruction or at least a loss of seal or air mix resulting in awakening and hypoxia. The rotation of the head is bound to happen, especially when dealing with a paediatric patient because of the size of the head is disproportionately larger to the body. Consequences of this can be dangerous leading to vomiting and aspiration. Complete respiratory obstruction can be very serious especially when the head of the patient is inaccessible during an operative procedure.

The reason for the rotation or mal positioning of the mask arises because the stiff airway tube is fixedly attached to the mask without allowing the mask part to lie in the desired position for its function when the head is rotated, flexed or extended.

Throughout the specification, the term “comprising” and its grammatical equivalents shall be taken to have an inclusive meaning unless the context of use indicates otherwise.

The present applicant does not concede that the prior art discussed in this specification forms part of the common general knowledge in Australia or elsewhere.

### BRIEF DESCRIPTION OF THE INVENTION

In a first aspect, the present invention provides a laryngeal mask for establishing an artificial airway in a patient, comprising a mask portion having a resilient conformable peripheral portion shaped such that the mask forms a seal with the larynx when the mask is positioned in the laryngopharynx, and an airway tube connected to or formed with the mask for passing gas to the larynx when the mask is properly inserted into the laryngopharynx, the mask portion including at least one region of reduced wall thickness or enhanced flexibility, and at least one region of increased wall thickness (relative to the reduced wall thickness) or enhanced stiffness (relative to the region of enhanced flexibility), the at least one region of increased wall thickness or enhanced stiffness having a larger longitudinal extent then a longitudinal extent of the at least one region of reduced wall thickness or enhanced flexibility.

In some embodiments, the at least one region of increased wall thickness or enhanced stiffness is located adjacent to the at least one region of reduced wall thickness or enhanced flexibility.

As the at least one region of increased wall thickness or enhanced stiffness has a longitudinal extent that is greater than the at least one region of reduced wall thickness or enhanced flexibility (and preferably is positioned adjacent to) the at least one region of increased wall thickness or enhanced stiffness provides a line of material that extends past the at least one region of reduced wall thickness or enhanced flexibility and beyond either end of the at least one region of reduced wall thickness or enhanced flexibility such that the at least one region of increased wall thickness or enhanced stiffness provide a line of material that can support and transmit a thrusting force applied to the laryngeal mask during insertion thereof into a patient. In this manner, the thrusting force applied by the anaesthetist facilitates movement of the mask portion downwardly and into the correct position in the patient. Furthermore, the at least one region of reduced wall thickness or enhanced flexibility located on the mask portion can also allow the mask to bend or deform at the required stages during insertion to minimise the risk of the mask portion becoming folded over upon itself during insertion. This facilitates correct insertion of laryngeal mask into the patient. Further, the that the at least one region of increased wall thickness or enhanced stiffness also helps to recoil the mask part back to its original position or shape when a bending force applied to the mask is released. This facilitates insertion of the distal end of the mask portion behind the larynx.

In one embodiment, the at least one region of reduced wall thickness comprises a ventral region or a dorsal region. In other embodiments, the at least one region of reduced wall thickness comprises two regions of reduced wall thickness. One of the regions of reduced wall thickness may be located on a ventral side of the mask portion and the other of the regions of reduced wall thickness may be located on a dorsal side of the mask portion.

In one embodiment, the at least one region of increased wall thickness or enhanced stiffness in the mask portion of the mask may be provided in a proximal end of the mask portion. The at least one region of increased wall thickness or enhanced stiffness may be located close to the region where the mask is connected to the airway tube or, in embodiments where the airway tube and mask portion are integrally formed with each other, close to the region where the mask portion merges into the airway tube.

In a second aspect, the present invention provides a laryngeal mask for establishing an artificial airway in a patient, comprising a mask portion having a resilient conformable peripheral portion shaped such that the mask forms a seal with the larynx when the mask is positioned in the laryngopharynx, and an airway tube connected to or formed with the mask for passing gas to the larynx when the mask is properly inserted into the laryngopharynx, the airway tube including at least one region of enhanced flexibility located at or near a distal end of the airway tube, the airway tube including a continuous region having increased stiffness (relative to the region of enhanced flexibility), the continuous region of increased stiffness extending from the distal end of the airway tube to a proximal end of the airway tube to thereby provide a continuous line of enhanced stiffness to transmit a pushing force therealong, the one or more regions of enhanced flexibility comprising one or more regions having an essentially identical wall thickness to the rest of the airway tube, but having a configuration that enhances flexibility.

In this aspect of the present invention, the one or more regions of enhanced flexibility comprise one or more regions having a similar wall thickness to the rest of the airway tube, but having a configuration that enhances flexibility. For example, the one or more regions of enhanced flexibility may be formed as a corrugated region or a bellows type region.

In the present invention, the at least one region of reduced wall thickness or enhanced flexibility facilitates bending or turning of the laryngeal mask in the required directions at the different sections of the patient's airway passage during insertion of the laryngeal mask into the patient. Furthermore, the provision of a continuous region of thicker wall section or increased stiffness that extends past the region(s) of reduced wall thickness or enhanced flexibility provides a continuous line through which thrust or a pushing force applied by the anaesthetist during insertion of the mask can act. This maintains the stiffness and the thrust for the laryngeal mask for moving in a forwards direction during insertion without folding or buckling.

In one embodiment, the at least one region of enhanced flexibility located at or near a distal end of the airway tube comprises a ventral region or a dorsal region. In other embodiments, the at least one region of enhanced flexibility comprises two regions of enhanced flexibility. One of the regions of enhanced flexibility may be located on a ventral side of the airway tube and the other of the regions of enhanced flexibility may be located on a dorsal side of the airway tube. The regions of enhanced flexibility may extend around part of a circumference of the airway tube. The regions of enhanced flexibility may extend along a longitudinal direction of the airway tube. The region or regions of enhanced flexibility suitably extend only along a portion of the longitudinal extent of the airway tube.

In other aspects of the present invention, the at least one region of reduced wall thickness or enhanced flexibility may be provided in a base plate of the mask. Accordingly, in a third aspect, the present invention provides a laryngeal mask for establishing an artificial airway in a patient, comprising a mask portion having a resilient conformable peripheral portion shaped such that the mask forms a seal with the larynx when the mask is positioned in the laryngopharynx, and an airway tube connected to or formed with the mask for passing gas to the larynx when the mask is properly inserted into the laryngopharynx, the mask portion including a base plate that, in use, is positioned dorsally of the resilient conformable peripheral portion, the base plate being formed with the airway tube or being connected to the airway tube, wherein the base plate includes at least one region of reduced wall thickness or enhanced flexibility, the base plate also including at least one region of thicker wall thickness or increased stiffness (relative to the at least one region of reduced wall thickness or enhanced flexibility, respectively), the region of thicker wall thickness or increased stiffness extending from a distal part of the base plate to a part of the base plate that joins with or merges into the airway tube.

In embodiments of the third aspect of the present invention, the base plate is configured so that it provides the desired flexibility to bend during insertion into the patient's airway whilst still providing a region of thicker wall thickness or increased stiffness that extends from a distal end of the base plate to the airway tube to thereby allow a thrusting force to be transferred therealong. This facilitates insertion of the laryngeal mask into the patient. The at least one region of reduced wall thickness or enhanced flexibility in the base plate may be located at a dorsal part of the base plate with the at least one region of thicker wall thickness or increased stiffness being located to one side of the at least one region of reduced wall thickness or enhanced flexibility. Suitably, the base plate has regions of thicker wall thickness or increased stiffness being located on either side of a dorsal region of reduced wall thickness or enhanced flexibility.

In another embodiment, the at least one region of reduced wall thickness or enhanced flexibility in the base plate may be located at a ventral part of the base plate with the at least one region of thicker wall thickness or increased stiffness being located to one side of the at least one region of reduced wall thickness or enhanced flexibility. Suitably, the base plate has regions of thicker wall thickness or increased stiffness being located on either side of the ventral region of reduced wall thickness or enhanced flexibility.

In another embodiment, one region of reduced wall thickness or enhanced flexibility in the base plate is located a dorsal part of the base plate and another region of reduced wall thickness or enhanced flexibility in the base plate is located a ventral part of the base plate, with the at least one region of thicker wall thickness or increased stiffness comprising two regions located circumferentially between the dorsal and ventral regions of reduced wall thickness or enhanced flexibility. In another embodiment of the present invention, the one or more regions of enhanced flexibility may comprise corrugated or fluted or bellows-shaped regions having the same wall thickness as the one or more regions of enhanced stiffness, with the one or more regions of intent stiffness being stiffer by virtue of not having a corrugated or fluted or bellows-shaped configurations.

The at least one region of increased wall thickness or enhanced stiffness, in addition to assisting in insertion of the device, may also help to maintain the airway cavity or passageway.

In some embodiments of the present invention, the resilient conformable peripheral portion shaped such that the mask forms a seal with the larynx comprises an inflatable portion. The inflatable portion may comprise an inflatable cuff, for example, has shown in British patent number GB 2,111,394. The resilient conformable peripheral portion may also be as described in U.S. Pat. No. 4,995,388. The entire contents of these documents are incorporated herein by cross reference.

Alternatively, the resilient conformable peripheral portion may comprise a non-inflatable portion. The resilient conformable peripheral portion may be as described in my international patent application numbers PCT/AU2004/001011 or PCT/AU20080001259, the entire contents of which are incorporated herein by cross reference.

In other embodiments, the resilient conformable peripheral portion may be similar to that present on a supraglottic airway device sold by Intersurgical Ltd, of Wokingham, United Kingdom. This supraglottic airway device includes a soft, non-inflatable cuff that fits snugly onto the perilaryngeal framework, mirroring the shape of the epiglottis, aryepiglottic folds, piriform fossae, peri-thyroid, peri-cricoid, posterior cartilages and spaces. Each receives an impression fit, thus supporting the seal by enveloping the laryngeal inlet. The seal created is sufficient for both spontaneously breathing patients and for IPPV.

The laryngeal mask of the present invention may comprise one or more cavities or tubes that, in use, have an opening that extends into the oesophagus of the patient. These one or more cavities or tubes may be provided for removing vomitus or other regurgitated material that may be expelled from the stomach of the patient during a procedure. Distal openings of the one or more cavities or tubes may be located such that, in use, they open in the oesophagus of the patient. The distal portion of the laryngeal mask may be provided with an extension that opens into the upper oesophagus. The extension may extend distally beyond the distal extent of the openings of the one or more cavities or tubes. In this embodiment, the extension acts as a protector that prevents tissues from being sucked into the one or more cavities or tubes. The extension may be made from a flexible material or a rigid material. In some embodiments, the extension may be surrounded or covered by any non-inflatable or inflatable cuff material or extension of the main cuff of the mask portion. The extension may be made from one or more loops that, in use, enter into the oesophagus. Other shapes may be used. In embodiments where the extension comprises two or more loops, one loop can be attached to the front of a tube or tubes entering into the oesophagus and the other can be attached across the tube or tubes. Suitably, the laryngeal mask may be provided with two cavities or tubes, with one cavity or tube being connected to suction and the other cavity or tube providing a vent opening. The extension may be made from a flexible material or rigid material.

In some embodiments, the extension may also facilitate insertion of the mask by helping the tip glide over the posterior pharyngeal wall without digging into the posterior pharyngeal wall which could cause a rift or a tear in the mucosa. When suction is applied to the cavities or tubes during insertion, the extension may also act to prevent the distal end of the tube(s) or cavity(ies) from sucking in the tissues of the pharyngeal wall and thus prevent the distal end of the tube(s) or cavity(ies) from becoming attached to the pharyngeal wall, which would otherwise prevent insertion.

In some embodiments, the laryngeal mask of the present invention may be provided with deformation means located on the mask, wherein the application of force to the deformation means causes elastic deformation of the device, thereby facilitating insertion of the device into the patient. Such embodiments may include features as described in my international patent application number PCT/AU2010/000341, the entire contents of which are here in incorporated by cross reference. The deformation means may assist in bending the mask to the required orientation during insertion.

In order to minimise the risk of the mask portion moving out of position should the head of the patient be rotated, the laryngeal mask may also include a joint between the mask portion and the airway tube, the joint allowing relative rotation of the airway tube relative to the mask portion.

Accordingly, in a fourth aspect, the present invention provides a laryngeal mask for establishing an artificial airway in a patient, comprising a mask portion having a resilient conformable peripheral portion shaped such that the mask forms a seal with the larynx when the mask is positioned in the laryngopharynx, and an airway tube connected to or formed with the mask for passing gas to the larynx when the mask is properly inserted into the laryngopharynx, characterised in that the laryngeal mask includes a joint adapted to permit relative rotation between the airway tube and the mask portion.

The joint may comprise a swivel joint or a hinge joint. The joint may comprise a ball and socket joint. The joint may comprise a joint that allows for rotation about at least two different axes.

The joint may be arranged such that a thrusting force applied to the airway tube may be transmitted through the joint to the mask portion of the device.

In some embodiments, the joint may also allow flexing and extension during insertion of the mask to facilitate the mask following the airway of the patient during the insertion procedure. The joint may also transmit thrust to push the mask down the patient's airway. The joint may also have a dorsi-flexion or hyperextension which will assist in maintaining the desired flex of the mask portion (typically a dorsi-flexed position) to help the mask portion slide behind the larynx.

The joint may be arranged such that restricted relative rotation between the mask portion and the airway tube is allowed. For example, the joint may be arranged such that a relative rotation of up to about 90° between the airway tube and the mask portion can occur. The joint may be provided with a stop member to cause restricted relative rotation. The joint may also allow flexion and extension.

In some embodiments, the mask portion may have a connector region, the connector region forming part of the joint or receiving part of the joint, the connector region including a region of reduced sidewall thickness or a region of enhanced flexibility or even a region where the sidewall is not present (either through the sidewall not being formed in that region during manufacture or by removal of the sidewall). The connector region may be positioned at or near a proximal end of the mask portion. The connector part of the joint may project out of the proximal part of the mask portion to connect to the airway tube. The connector can be a separate connector to increase the flexibility of the joint.

In another aspect, the present invention provides a laryngeal mask for establishing an artificial airway in a patient, comprising a mask portion having a resilient conformable peripheral portion shaped such that the mask forms a seal with the larynx when the mask is positioned in the laryngopharynx, and an airway tube connected to or formed with the mask for passing gas to the larynx when the mask is properly inserted into the laryngopharynx, wherein the mask portion includes a dorsal surface, characterised in that a line extending from a proximal part to a distal part of a dorsal-most part of the dorsal surface extends generally parallel to a longitudinal axis of a distal most part of the airway tube, or the line extending from a proximal part to a distal part of a dorsal-most part of the dorsal surface extends towards a ventral region in a direction from the proximal to distal part of that line.

In a further aspect, the present invention provides a laryngeal mask for establishing an artificial airway in a patient, comprising a mask portion having a resilient conformable peripheral portion shaped such that the mask forms a seal with the larynx when the mask is positioned in the laryngopharynx, and an airway tube connected to or formed with the mask for passing gas to the larynx when the mask is properly inserted into the laryngopharynx, wherein the mask portion includes a base plate, the base plate having a dorsal surface, characterised in that a line extending from a proximal part to a distal part of a dorsal-most part of the dorsal surface extends generally parallel to a longitudinal axis of a distal-most part of the airway tube or the line extends at an angle to the longitudinal axis of a distal most part of the airway tube such that the line extends distally and ventrally from a proximal end of the line relative to the longitudinal axis of a distal most part of the airway tube.

One currently available laryngeal mask, the Classic Laryngeal Mask as manufactured and provided by the Laryngeal Mask Company, has a mask portion in which the corresponding line extends dorsally and distally from a proximal end of the line. The present inventor has surprisingly found that making the modification as outlined in the immediately two preceding paragraphs above effectively changes the angle of the base plate or mask portion relative to the airway tube and this can significantly ease and facilitate insertion of the mask into the airway of a patient. With this modification, the additional features of other aspects of this invention may not necessarily be required to achieve ease of insertion into the airway of a patient.

In a further aspect, the present invention provides a method for manufacturing a laryngeal mask, the laryngeal mask including a mask portion defining a chamber, and an inflatable cuff extending around the chamber, the chamber being in fluid communication with an airway tube such that gases supplied from the airway tube pass into the chamber and thereafter into the larynx of a patient when the mask is in use, the method comprising the steps of:

a) forming the mask portion such that a skirt extends from the mask portion, the mask portion having at least one lip spaced from a base of the skirt or two spaced lips;
b) inserting a free end of the skirt into a space defined between the at least one spaced lip and a base of the skirt or between the two spaced lips; and
c) retaining the free end of the skirt between the at least one spaced lip and the base of the skirt or between two spaced lips such that the skirt defines a substantially closed outer surface and can function as an inflatable cuff.

In one embodiment, a circumferential length of the free end of the skirt is less then a circumferential length of the space between the at least one spaced lip and the base of the skirt or between two spaced lips. In this embodiment, it may be necessary to stretch the circumference of the free end of the skirt to fit the skirt into the space. This will tend to retain the free end of the skirt in the space. This, in turn, allows a smaller amount of glue or other adhesive to be used. It may even allow no glue or adhesive to be required.

Suitably, the free end of the skirt is glued in position between the two spaced lips. However, other means of fixing the free end of the skirt between the two spaced lips, such as ultrasonic welding, or use of a suitable double sided adhesive tape, may also be used. Alternatively, in some embodiments, a friction fit between the spaced lips and the free end of the skirt may be used to retain the free end of the skirt in place. As a further alternative, the two spaced lips may be resilient lips and the cuff may be formed by inserting the free end of the skirt between the two spaced lips, with the resilience of the lips holding the free end of the skirt in position.

The mask portion may be formed by a moulding process. Suitable moulding processes may include injection moulding or rotamoulding. The moulding process forms the chamber, which will typically be part of a base plate of the mask portion. The moulding process can also be used to form the skirt and the at least one lip or the two spaced lips. Suitably, the chamber, the skirt and the at least one lip or the two spaced lips are formed in a single moulding process.

The lips are not particularly limited in their shape or their size or number. It will be appreciated that the space between the two lips will normally be larger than the thickness of the free end of the skirt. This enables the free end of the skirt to be inserted into the space between the two lips. However, if one or both of the lips are made from a resilient material, it is not necessary that the space between the lips be larger than the thickness of the free end of the skirt because the free end of the skirt can be inserted between the lips by forcing the one or both resilient lips apart from each other.

The two spaced lips or the at least one lip and the base of the skirt desirably circumscribe a shape that forms part of the shape of the cuff in the completed mask.

At this stage of the moulding process, the mask portion may be removed from the mould in a simple fashion. The mask portion will typically include a base plate having an airway extension (the airway extension is ultimately joined to the airway tube). When the mask portion is removed from the mould, the skirt extends away from the mask portion. Desirably, the skirt extends around the mask portion, with a free end of the skirt being located away from the mask portion.

In order to complete manufacture of the mask portion, the free end of the skirt is inserted into the space between the two lips and the free end of the skirt is fixed in place. This results in the skirt defining the inflatable cuff. Suitably, the skirt includes an inlet that defines a passage for receiving inflation air from an inflation tube so that the inflatable cover can be selectively inflated and deflated. The inflation tube may be permanently connected to the inlet of the cuff.

By positioning the free end of the skirt between the base of the skirt and the at least one lip or between the two spaced lips, a smaller amount of glue or other fixing means may be used to hold the free end of the skirt in place to form the inflatable cuff. As a result, it is possible to make the mask portion with greater flexibility than prior art masks that have inflatable cuffs. In this regard, prior art masks that have inflatable cuffs require significantly larger amounts of glue to form the cuff than is required in this aspect of the present invention. This large amount of glue forms a line of stiffening around the cuff which, in turn, stiffens the mask portion of the laryngeal mask.

In another aspect, the present invention also encompasses a laryngeal mask including a mask portion defining a chamber, and an inflatable cuff extending around the chamber, the chamber being in fluid communication with an airway tube such that gases supplied from the airway tube pass into the chamber and thereafter into the larynx of a patient when the mask is in use, wherein the inflatable cuff is formed from a skirt extending from the mask portion and having an end of the skirt being positioned between and retained between at least one lip and a base of the skirt or between two spaced lips.

The at least one lip or the two spaced lips may extend from a ventral part of the chamber.

### BRIEF DESCRIPTION OF THE DRAWINGS

FIG. 1 is a perspective view of a laryngeal mask in accordance with one embodiment of the present invention;

FIG. 2 is a side cross sectional view of the laryngeal mask shown in FIG. 1;

FIG. 3 is a side view of a laryngeal mask in accordance with another embodiment of the present invention;

FIG. 4 is a side view of the laryngeal mask in accordance with another embodiment of the present invention;

FIG. 5 is a side view of a laryngeal mask in accordance with yet another embodiment of the present invention;

FIG. 6 is a perspective view of a laryngeal mask in accordance with another embodiment of the present invention;

FIG. 7 is a cross-sectional end view of the laryngeal mask shown in FIG. 6 and taken along the line A-A of FIG. 6;

FIG. 8 is a side view of a laryngeal mask in accordance with a further embodiment of the present invention;

FIG. 9 is a side view of a laryngeal mask in accordance with another embodiment of the present invention;

FIG. 10 is a schematic view of a laryngeal mask properly positioned within the laryngopharynx of the patient;

FIG. 11 is a schematic view of a laryngeal mask that has folded over on itself during insertion into a patient;

FIG. 12 is a side view of a laryngeal mask in accordance with another embodiment of the present invention;

FIG. 13 is a side view of a laryngeal mask in accordance with yet another embodiment of the present invention;

FIG. 14 is a perspective view of a laryngeal mask in accordance with a further embodiment of the present invention;

FIG. 15 is a side view of the laryngeal mask shown in FIG. 14;

FIG. 15A shows a cross sectional view taken along line A-A of FIG. 15;

FIG. 15B shows a cross-sectional end view of the airway tube taken along line B-B of FIG. 15;

FIG. 16 is a view of a part of a laryngeal mask suitable for use in embodiment of the present invention;

FIG. 17 is a view of another part of a laryngeal mask suitable for use with the part shown in FIG. 16;

FIG. 18 is a side view of a laryngeal mask in accordance with another embodiment of the present invention;

FIG. 19 is a side view of the laryngeal mask in accordance with yet another embodiment of the present invention;

FIG. 20 is a side view, partly in cross-section, of the laryngeal mask shown in FIG. 18;

FIG. 21 is a side view of a laryngeal mask in accordance with a further embodiment of the present invention;

FIG. 22 is a side view of part of the laryngeal mask shown in FIG. 21;

FIGS. 23 and 24 shows side views of components of a joint suitable for use in the laryngeal mask shown in FIG. 21;

FIG. 25 is a view of the assembled joint made from the components shown in FIGS. 23 and 24;

FIG. 26 is a side view (showing hidden detail of the joint) of the mask shown in FIG. 21;

FIG. 27 is a side view of a part suitable for use in a laryngeal mask in accordance with another embodiment of the present invention;

FIG. 28 is a side view of laryngeal mask incorporating the part shown in FIG. 27;

FIGS. 29 to 32 show various views of a joint suitable for use in a laryngeal mask in accordance with the embodiment of the present invention;

FIG. 33 shows components of the joint apart and in line for assembly;

FIG. 34 shows the assembled joint in cross-section;

FIGS. 35, 36 and 37 show cross-sectional views of airway tubes suitable for use in embodiments of the present invention;

FIG. 38 shows a view of some assembly details of a laryngeal mask in accordance with the present invention;

FIG. 39 shows a perspective view of a laryngeal mask in accordance with a further embodiment of the present invention;

FIG. 40 shows a perspective view of a laryngeal mask in accordance with a further embodiment of the present invention;

FIG. 41 shows a side view of a laryngeal mask in accordance with another embodiment of the present invention;

FIG. 42 shows a dorsal view of a laryngeal mask in accordance with another embodiment of the present invention;

FIG. 43 shows a perspective view from the ventral side of the mask shown in FIG. 42;

FIGS. 44 and 45 show a perspective view from a ventral side and a dorsal view, respectively, of a laryngeal mask in accordance with yet another embodiment of the present invention;

FIG. 46 shows a side view of a laryngeal mask in accordance with yet another embodiment of the present invention;

FIG. 47 shows a side view of a laryngeal mask in accordance with a further embodiment of the present invention;

FIG. 48 shows a perspective view from a dorsal side of a laryngeal mask in accordance with another embodiment of the presentation;

FIG. 49 shows a perspective view looking from a distal end of the mask shown in FIG. 48 and showing a cross-section taken along line A-A of FIG. 48;

FIG. 50 is a further view of the laryngeal mask shown in FIGS. 48 and 49;

FIG. 51 shows a side view of a laryngeal mask in accordance with another embodiment of the present invention;

FIG. 52 shows a cross sectional side view of a laryngeal mask in accordance with a further embodiment of the present invention;

FIG. 53 shows a side view of a laryngeal mask in accordance with another embodiment of the present invention;

FIGS. 54 and 55 show laryngeal masks that have a number of similarities to the masks showing in FIGS. 51 to 53, but with slightly different arrangements of features thereof;

FIG. 56 shows a cross sectional side view of a laryngeal mask in accordance with a further embodiment of the present invention;

FIGS. 57 and 58 shown perspective views of another embodiment of a laryngeal mask in accordance with the present invention. FIG. 57 shows the completed mask with the airway tube attached (part of the airway tube is shown in cross-section) whilst FIG. 58 shows the mask portion with the airway tube not attached;

FIG. 59 shows a view of a laryngeal mask in accordance with another embodiment of the present invention;

FIG. 60 shows a mask portion suitable for use in a laryngeal mask in accordance with a further embodiment of the present invention. FIG. 60 also shows additional joint components for use in the laryngeal mask;

FIG. 61 shows a view of the assembled joint components shown in FIG. 60;

FIG. 62 shows an airway tube being connected to the mask portion shown in FIG. 60;

FIG. 63 shows a ventral view of a laryngeal mask in accordance with another embodiment of the present invention;

FIG. 64 shows a cross-sectional view taken along line A-A of FIG. 63;

FIG. 65 shows a laryngeal mask in accordance with another aspect of the present invention;

FIG. 66 shows a laryngeal mask in accordance with another aspect of the present invention;

FIG. 67 shows a laryngeal mask in accordance with another aspect of the present invention;

FIG. 68 shows a laryngeal mask in accordance with another aspect of the present invention;

FIG. 69 shows a view of an alternative connector that can be used to connect a mask portion to an airway tube;

FIG. 70 shows a side view of a mask portion for use in a laryngeal mask for establishing an artificial airway in a patient;

FIG. 71 shows a cross sectional view of a conventional prior art laryngeal mask portion;

FIG. 72 shows a cross sectional view of a mask portion in accordance with an embodiment of the present invention following formation, such as by moulding, of the mask portion, but before completion of the inflatable cuff;

FIG. 73 shows a cross sectional view of part of the mask portion shown in FIG. 72 with the inflatable cuff being completed;

FIG. 74 shows a cross sectional view of a mask portion in accordance with an embodiment of the present invention following formation, such as by moulding, of the mask portion, but before completion of the inflatable cuff;

FIG. 75 shows a cross sectional view of part of the mask portion shown in FIG. 72 with the inflatable cuff being completed;

FIG. 76 shows a cross sectional view of a mask portion in accordance with an embodiment of the present invention following formation, such as by moulding, of the mask portion, but before completion of the inflatable cuff; and

FIG. 77 shows a cross sectional view of part of the mask portion shown in FIG. 76 with the inflatable cuff being completed.

### DETAILED DESCRIPTION OF THE DRAWINGS

It will be understood that the drawings have been provided for the purposes of illustrating preferred embodiments of the present invention. Therefore, the skilled person will appreciate that the present invention should not be considered to be limited solely to the features as shown in the drawings.

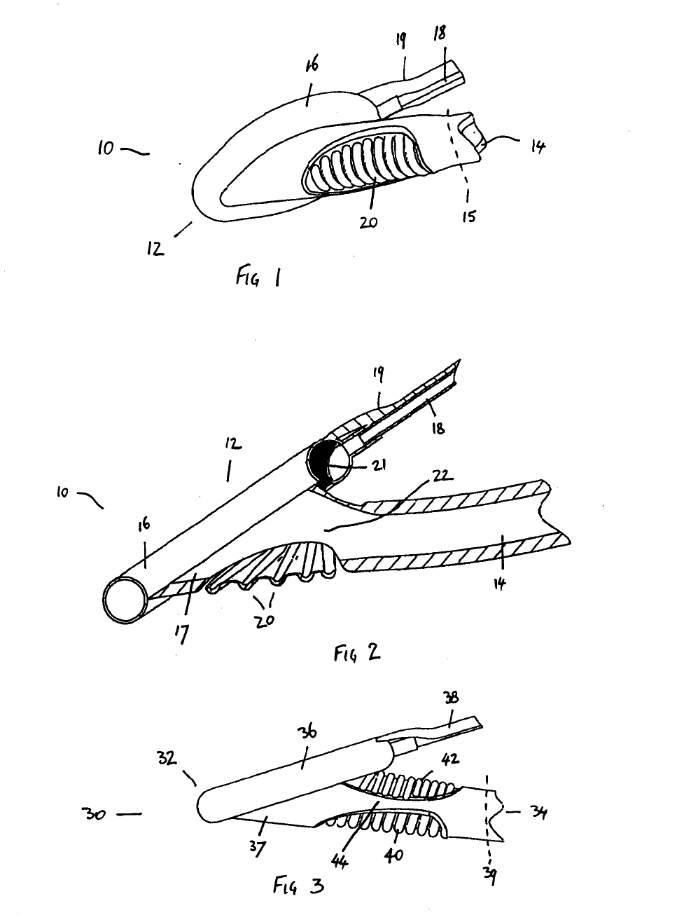

FIGS. 1 and 2 show a laryngeal mask 10 that comprises a mask portion, generally denoted by reference numeral 12, and an airway tube 14. The mask portion 12 and airway tube 14 may be integrally formed or they may be formed from separate components that are subsequently joined together. The mask portion 12 and the airway tube 14 will typically be joined together or merge at or near the region shown by dashed line 15 in FIG. 1. The mask portion 12 has an airway extension 17 where a dorsal part of the mask portion 12 changes shape so that it can either connect to the airway tube 14 or, in embodiments where the mask 10 is integrally formed, merge into the airway tube 14.

The mask portion 12 includes an inflatable cuff 16 that is selectively inflated and deflated via a small inflation tube 18. The inflatable cuff may be of conventional construction. The airway tube 14 is suitably made from a relatively rigid plastic material. In order to facilitate bending or flexing of the laryngeal mask during insertion into the patient, the proximal end of the mask portion 12 may be provided with a plurality of corrugations 20. The corrugations 20 provide a region of enhanced flexibility located near the proximal end of the mask portion and distally from the distal end of the airway tube. As can be shown from FIGS. 1 and 2, the corrugations 20 extend part way around the circumference of the airway extension 17 of the mask portion 12. Similarly, the corrugations 20 extend along a longitudinal extent of the airway extension 17 of the mask portion 12. As can be seen from FIG. 2, there is a region, denoted by reference numeral 22, of the airway extension 17 of the mask portion 12 that is positioned adjacent to the corrugations 20 and extends along the longitudinal extent of the corrugations 20. The region 22 has a wall thickness that corresponds to the general wall thickness of airway extension 17 of the mask portion 12 (and may also be of a similar thickness to the thickness of the airway tube 14). Therefore, region 22 provides a continuous line of relatively thick wall thickness (and thus relatively stiff material) that extends from a proximal end located proximally of the proximal end of the corrugations 20 to a distal end that is located distally of the distal extent of the corrugations 20. This continuous line of relatively stiff material is capable of transferring a thrusting or pushing force applied by the anaesthetist to a proximal end of the airway tube 14 down to the mask portion 12. Consequently, insertion of the mask is facilitated. Moreover, the corrugations 20 provide a region of enhanced flexibility such that the laryngeal mask 10 can flex at the appropriate places and times during insertion to further facilitate insertion of the laryngeal mask 10 into a patient. Region 22 also assists in returning the mask 10 to its desired orientation when the mask has been inserted into a patient and the forces associated with the insertion process have been removed. Typically, the mask should flex dorsally towards or at the end of insertion and the region 22 of enhanced stiffness assists in ensuring that this flexion does occur.

The corrugations 20 may be formed as part of a moulding process used to manufacture the laryngeal mask 10. Alternatively, the corrugations 20 may be formed by forming the laryngeal mask 10, forming openings in the region of the corrugations 20 and closing the openings with a thin plastic or thin membrane.

In order to assist in bending the mask to a desired shape during insertion of the mask into the airway of the patient, the mask is also provided with a tongue 19 that terminates in a septum 21. The septum 21 may extend through the inflatable cuff 16 and be attached to the base plate of the mask portion 12. The septum 21 assists in firmly attaching the tongue 19 to the mask portion 12. Furthermore, as the septum 21 is connected to the mask portion across the width of the septum, any pulling force applied to the tongue 19 is more effectively transferred to the mask portion 12. It will be understood that all embodiments of the present invention that include a deformation means may have the deformation means attached to the mask portion via a septum.

FIG. 3 shows a laryngeal mask 30 in accordance with another embodiment of the present invention. The laryngeal mask 30 includes a mask portion, generally referred to by reference numeral 32 and an airway tube 34. The mask portion 32 includes an inflatable cuff 36 that can be selectively inflated and deflated by inflation tube 38. The mask portion 32 includes an airway extension 37 that connects to or merges with the airway tube 34 at or near a region denoted by dashed line 39 in FIG. 3.

The laryngeal mask 30 also includes a dorsal region 40 in which the wall section is formed by a relatively thin wall and a ventral region 42 in which the wall section is also formed by a relatively thin wall. Regions 40 and 42 extend part of the way around the circumference of the airway tube 44 and extend to a longitudinal extent along a longitudinal direction of the airway extension 37. Positioned between regions 40 and 42 is a region 44 of relatively thick wall section. Region 44 may be simply formed as a continuation of the airway extension 37. As can be seen from FIG. 3, region 44 provides a bridge or a continuous line of relatively thick wall section extending from a proximal end of the mask portion 32 to a distal end of the mask portion 32. Region 44 allows a thrust force or pushing force to be transmitted to the mask portion 32 during insertion of the laryngeal mask 30 into a patient. The regions 40, 42 of relatively thin wall section allow the mask to flex in the vicinity of those regions 40, 42 to thereby facilitate bending of the mask at the appropriate stages during insertion of the mask into the patient.

The regions 40 and 42 may be formed as thin-walled regions during moulding. Alternatively, the regions 40, 42 may be formed by excising material from the laryngeal mask 30 and subsequently covering the openings thus formed with a thin material or in membrane. This will ensure that the airway extension 37 of mask portion 32 is fully sealed. In some embodiments, the regions of relatively thin wall thickness may be replaced by corrugations or a concertina-type material.

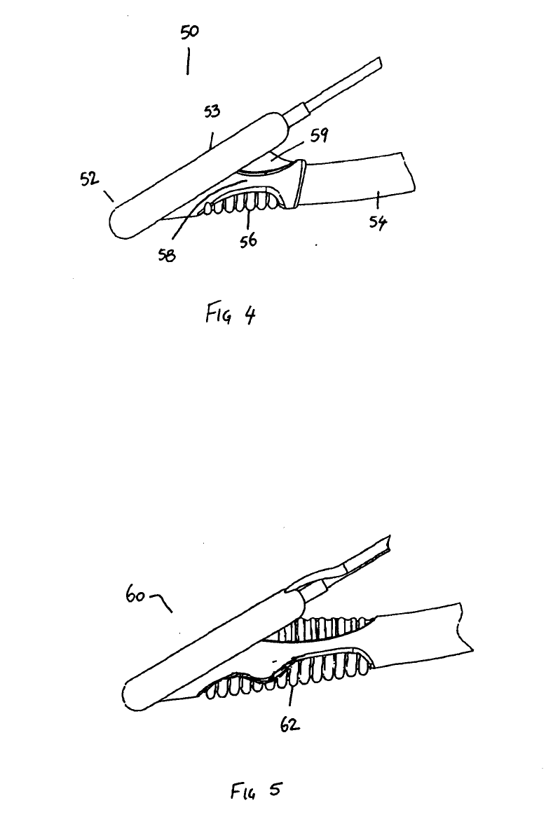

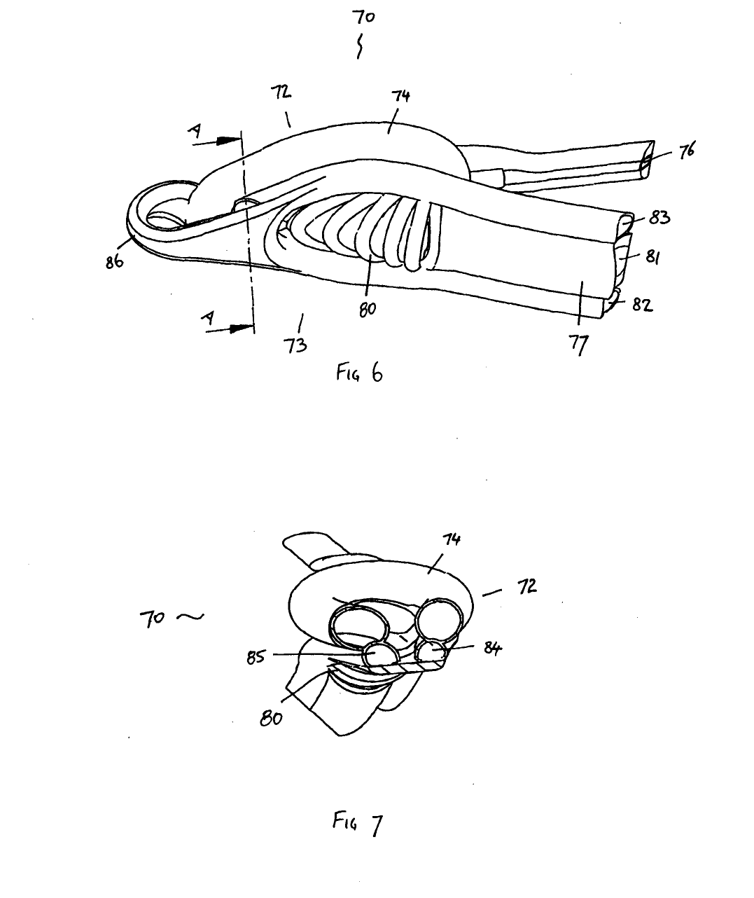

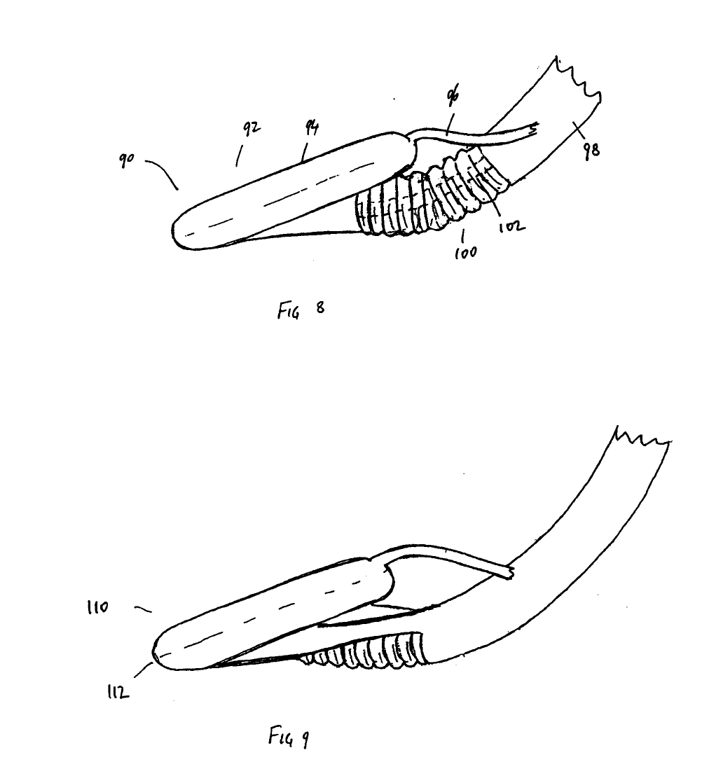

FIG. 4 shows a laryngeal mask 50 in accordance with a further embodiment of the present invention. The laryngeal mask 50 includes a mask portion 52 having an inflatable cuff 53, and an airway tube 54. The mask 50 also includes a dorsal region 56. Dorsal region 56 comprises a region of corrugations. Region 56 is located near a proximal end of the mask portion 52. A region 58 of enhanced stiffness (when compared to the region 56) extends longitudinally and adjacent to the region 56 of corrugations. It may also be possible to include a relatively thin wall section or membrane 59 on the ventral side of the airway tube 54.

FIG. 5 shows a laryngeal mask 60. The laryngeal mask 60 has a number of features in common with the mask shown in FIG. 3. Where laryngeal mask 60 differs from the mask shown in FIG. 3 is that the dorsal region 62 that has a relatively thin wall extends further in the longitudinal direction than the dorsal thin wall region 40 of the mask shown in FIG. 3. The dorsal region 62 shown in FIG. 5 extends almost to the distal end of the laryngeal mask 60.

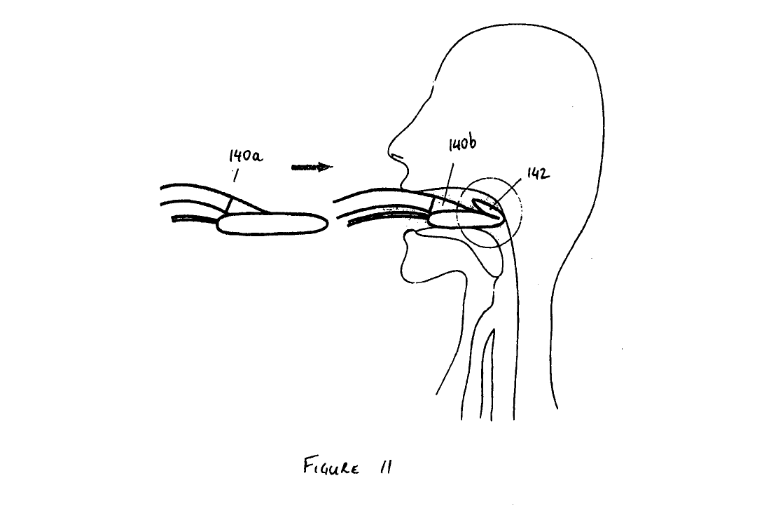

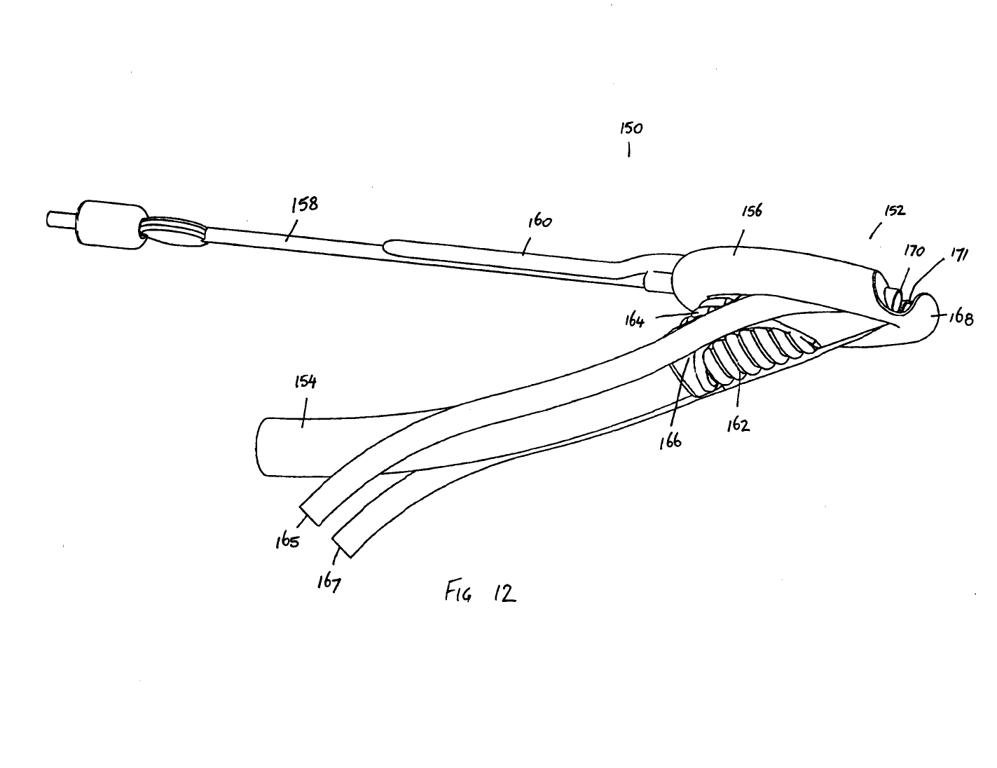

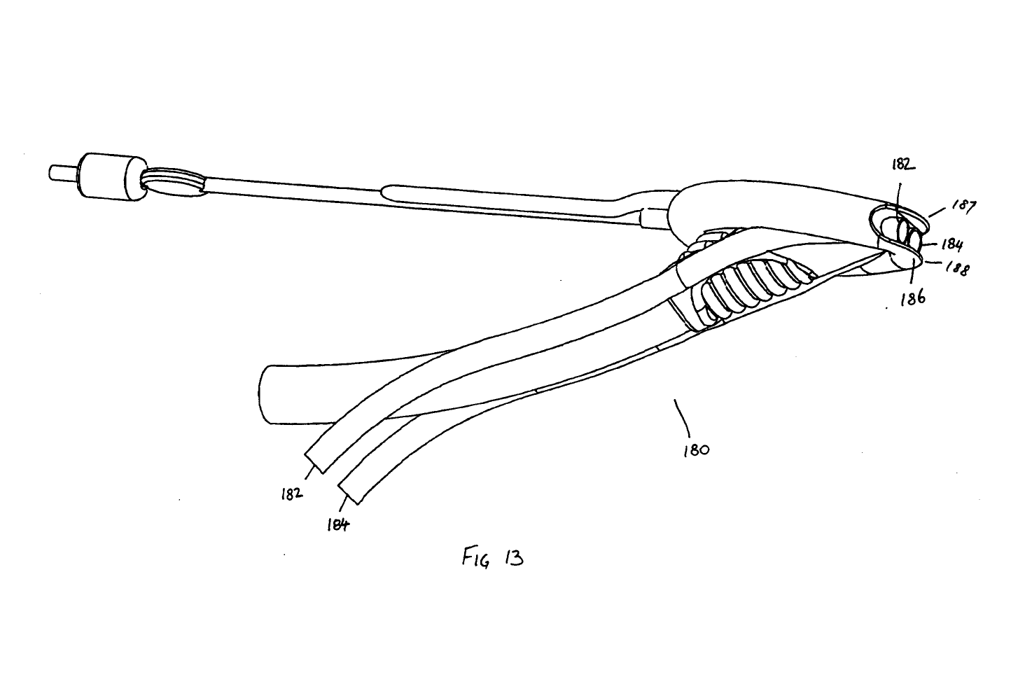

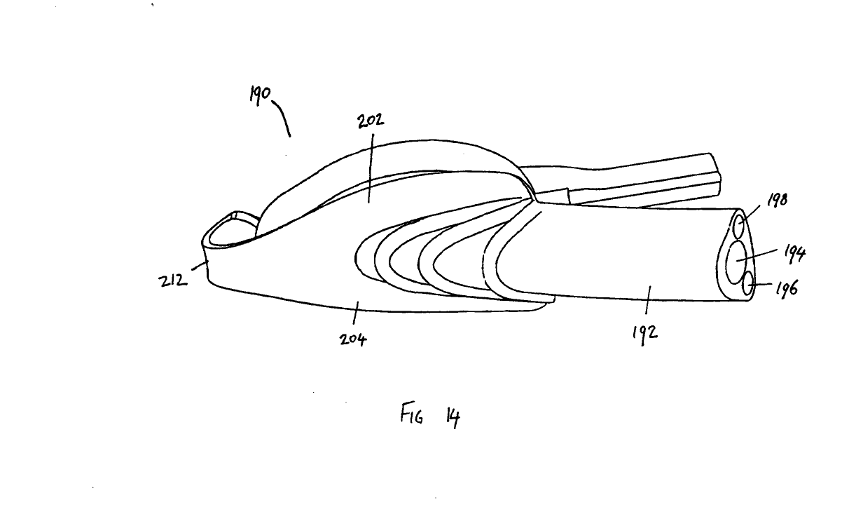

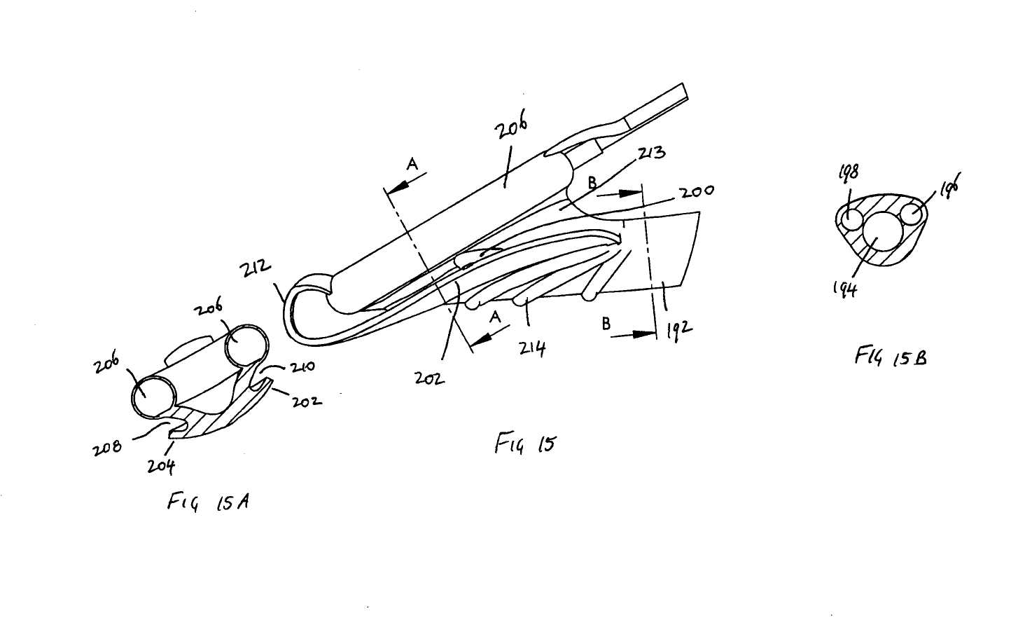

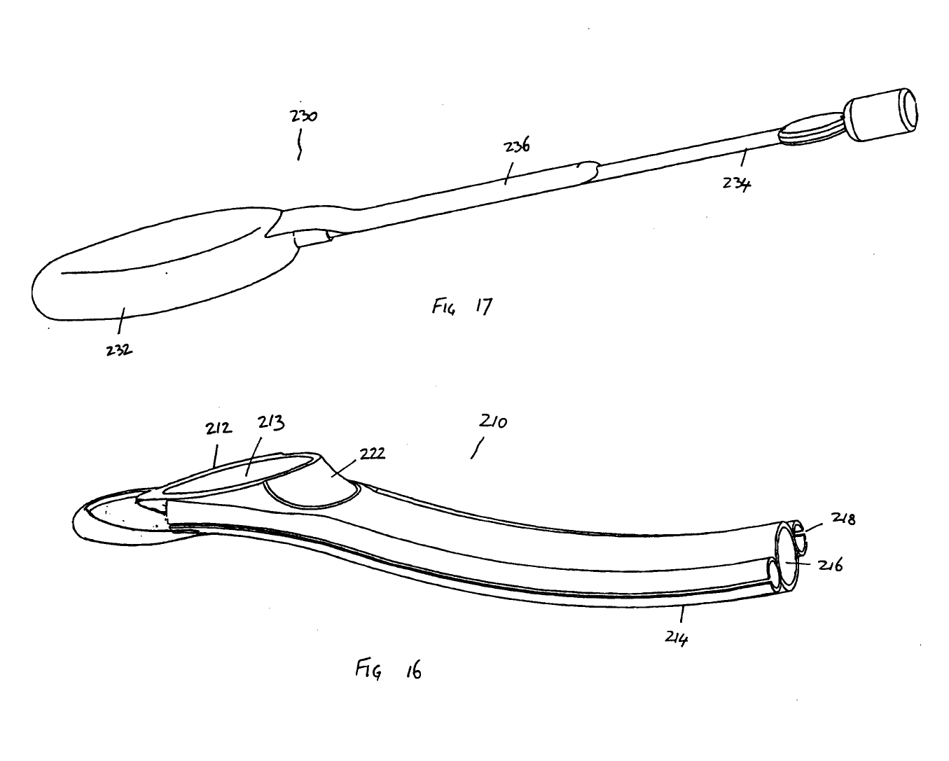

FIGS. 6 and 7 show a laryngeal mask 70. The laryngeal mask 70 has a mask portion 72 that includes an inflatable cuff 74 that can be selectively inflated and deflated by inflation tube 76. The laryngeal mask 70 includes an airway tube 77. Mask portion 72 includes an airway extension 73 that joins with airway tube 77. Airway extension 73 has a soft membrane part in a ventral region thereof (not shown) and a corrugated region 80. The airway tube 77 includes a central airway tube 81 for supplying ventilation gases to the larynx/lungs of the patient. The airway tube 81 is also provided with two side tubes 82, 83 that have openings 84, 85 at the dorsal distal end of the mask. The laryngeal mask 70 also includes an extension or projection 86 that, in use, extends into the oesophagus of the patient. The distal ends of tubes 82, 83 also extend into the oesophagus or are located near the oesophagus when the mask is properly inserted into the patient such that any vomitus or gastric juices may enter the openings 84, 85. Advantageously, suction can be applied to one of tubes 82, 83 and the other of tubes 82, 83 can act as a vent tube in order to allow the removal of vomitus or gastric juices from the oesophagus. The extension or projection 86 acts to space the tissues of the oesophagus away from the openings 84, 85 to thereby prevent or minimise the likelihood of the tissues of the oesophagus becoming sucked into the openings 84, 85 in the event that suction is applied to the relevant tube 82, 83. The extension or projection 86 also assists in keeping the oesophagus slightly open when the laryngeal mask 70 is properly inserted, thereby minimising the likelihood that a large surge of vomitus will surge past a closed oesophagus.

In common with the embodiments shown in FIGS. 1 to 5, the laryngeal mask 70 shown in FIGS. 6 and 7 includes a region of thicker wall section/enhanced stiffness when compared to the regions 78, 80. In FIGS. 6 and 7, the regions of thicker wall section/enhanced stiffness are positioned underneath the tubes 82, 83.

FIG. 8 shows a laryngeal mask 90 in accordance with another embodiment of the present invention. The laryngeal mask 90 includes a mask portion 92 having an inflatable cuff 94. The inflatable cuff 94 can be selectively inflated and deflated via small tube 96. The laryngeal mask 90 includes an airway tube 98. A concertina membrane 100 is attached over the airway tube 98. The concertina portion may have a wall thickness that is generally similar to the wall thickness of the airway tube 98. Underneath the concertina membrane 100, at least a part of the wall of the airway tube has been removed to thereby provide regions of enhanced flexibility. As shown in dotted outline 102 in FIG. 8, a region of the wall of airway tube 98 is not removed and it extends underneath the concertina membrane 100 to thereby provide a continuous line of relatively enhanced stiffness to thereby facilitate transmittal of a pushing force to the mask portion and 92.

FIG. 9 shows a laryngeal mask 110 that has a number of similarities to the laryngeal mask 50 shown in FIG. 4. Where the laryngeal mask 110 differs from the mask 50 shown in FIG. 4 is that the distal end 112 of the mask portion of the laryngeal mask 110 extends further in a distal direction then the distal portion of the mask portion 52 shown in FIG. 4.

FIG. 10 shows a laryngeal mask 120 properly inserted into a patient. As can be seen, the distal end 122 of the mask 120 extends into the oesophagus 124. The airway tube 126, which is in fluid communication with an outlet that opens in the interior of the mask portion 128, passes anaesthetic gases or ventilation gases into the larynx 130 of the patient.

FIG. 11 shows a schematic diagram of a laryngeal mask 140a ready for insertion into a patient's airway. As can be seen from FIG. 11, as the mask is moved to the position shown at 140b, the distal end of the mask portion 142 has been caused to fold back upon itself, thereby resulting in the laryngeal mask 140 not being properly inserted into the patient. In the embodiment shown in FIGS. 1 to 9, the presence of the more flexible regions or thinner wall regions enables the laryngeal mask to bend and flex when in the vicinity of those regions so that the mask more easily moves to a desired orientation to enable the mask to move around and past the complex curves that are encountered during insertion of the laryngeal mask into the patient's airway. Similarly, the regions of enhanced stiffness/thicker wall section enable the pushing or thrusting force applied to the airway tube by the anaesthetist to be transferred to the mask portion of the laryngeal mask. This also facilitates insertion of the mask.

FIG. 12 shows a laryngeal mask 150 having a mask portion 152 and an airway tube 154. Mask portion 152 is manufactured as a separate part to airway tube 154 and the mask portion 152 and airway tube 154 are subsequently joined together. The mask portion 152 includes an inflatable cuff 156. An inflation tube 158 is used to selectively inflate and deflate the inflatable cuff 156. The mask portion 152 also has a deformation means 160 attached thereto. The deformation means comprises a tongue of material that is attached to a proximal end of the cuff 156. The tongue 160 can be pulled or manipulated by the anaesthetist during insertion of the laryngeal mask 150 into a patient so that the laryngeal mask 150 is bent or flexed to the appropriate degree at the appropriate stages during insertion.

To facilitate bending of the mask, corrugated regions 162, 164 are provided in the base plate of the mask portion 152. A region of relatively enhanced stiffness or increased wall thickness 166 is also provided. These regions are similar to the similar regions as shown in the embodiments of FIGS. 1 to 9.

The mask 150 also includes a projection 168 that extends from the distal end of the mask portion 152. Projection 168, in use, extends into the oesophagus of the patient. The mask 150 also includes tubes 165, 167 having outlets 170, 171. Outlets 170, 171 can receive vomitus or gastric material from the oesophagus and it can be used to remove or drain the vomitus or gastric material from the oesophagus. The downwardly extending flap or projection 168 also provides for ease of insertion of the mask and assists in preventing the distal end of the mask digging in to the mucosa at the back of the patient's throat. It will be appreciated that rifts or tears could be formed in the mucosa if the distal end of the mask digs into the mucosa during insertion of the mask into the patient's airway.

FIG. 13 shows a laryngeal mask 180 that is very similar to the laryngeal mask shown in FIG. 12. However, the laryngeal mask 180 shown in FIG. 13 includes two gastric tubes 182, 184 for removing vomitus or gastric juices from the oesophagus. One of the tubes 182 or 184 could also be used to provide venting gas to the oesophagus if suction is applied to the other tube. The shape of the distal projection 186 also differs to that shown in FIG. 12. In particular, projection 186 comprises a ventral projection 187 and a dorsal projection 188, both of which extend from the distal end of the mask portion and extend above and below, respectively, the distal openings of tubes 182, 184. This prevents tissues from the patient's airway or oesophagus being drawn into the distal openings 182, 184 by suction being applied to the tubes 182 and 184. It will be appreciated that suction may be applied to one of the tubes 182, 184 and the other of those tubes may be used to provide a vent to prevent an undesirable build-up of suction at the distal end of the laryngeal mask during use.

The projections 187, 188 also assists in keeping the oesophagus open during use, at least to a small extent.

FIGS. 14 and 15 show a laryngeal mask 190 that is somewhat similar to the mask is shown in FIGS. 6 and 7. FIG. 15A shows a cross sectional view taken along line A-A of FIG. 15. FIG. 15B shows a cross-sectional end view of the airway tube taken along line B-B of FIG. 15. The airway tube 192 includes a passageway 194 for supplying ventilation gases or anaesthetic gases to the larynx/lungs of the patient. Two smaller passageways 196, 198 are also formed in the airway tube 194. These passageways have an outlet 200 and another outlet not shown, respectively, (see FIG. 15) that is positioned underneath laterally extending wings 202, 204. Laterally extending wings 202, 204 define, with the inflatable cuff 206, passageways 208, 210 that extends along most, if not all, of the longitudinal extent of the mask portion of the laryngeal mask 190. Oesophageal projection 212 is also provided. Oesophageal projection 212 is on the form of a loop of material extending from a dorsal part of the distal end of the mask portion to a ventral part of the distal end of the mask portion. In use, oesophageal projection 212 extends into the oesophagus of the patient. Should any gastric juices or vomitus come up out of the oesophagus, they can pass along passageways 208, 210 and be removed through passageways 196, 198.

Also shown in FIGS. 14 and 15 are the thin membrane ventral region 213 and the corrugated dorsal region 214 that provide appropriate flexibility to the mask portion of the laryngeal mask 190. Although not clearly shown in FIGS. 14 and 15, one or more regions of thicker wall thickness or enhanced stiffness, similar to those as described with reference to FIGS. 1 to 9, are also provided in the laryngeal mask 190.

FIG. 16 shows a view of a part of the laryngeal mask. The part 210 shown in FIG. 16 includes a base plate 212 and an airway portion 214. Airway portion 214 includes an airway passage 216 for passing ventilating gases or anaesthetic gases to the lungs of the patient. The part 210 also includes passageways 218, 220 that, in use, are placed in fluid communication with the oesophagus. The part 210 is suitably made from a relatively rigid plastic material.

The part 210 includes a ventral region 222 that has enhanced flexibility or a thinner wall section. The part 210 also includes a dorsal region (not shown) that provides a region of a thinner wall thickness or a region of enhanced flexibility.

FIG. 17 shows another part of the laryngeal mask that may be affixed to the part 210 shown in FIG. 16. The part 230 shown in FIG. 17 includes the cuff portion 232, and inflation tube 234 and a deformation means/tongue 236. In order to manufacture a laryngeal mask from the part 210 and the part 230, the cuff 232 of part 230 is positioned over the opening 213 formed in the base plate 212. The cuff 232 is then joined or affixed to the base plate 212.

Although the embodiments of the invention shown in FIGS. 1 to 17 describe the use of inflatable cuff, it will be appreciated that it is not necessary to utilise an inflatable cuff in order to obtain a seal around the larynx of the patient. Indeed, the cuff may comprise inflatable cuff, a non-inflatable solid cuff or an expandable but non-inflatable cuff. In other embodiments, the cuff may comprise a non-inflatable but expandable cuff. For example, the cuff may extend due to ventilation gases passing into the cuff, as described in my international patent application number PCT/AU2008/001259. The person skilled in the art will appreciate that there may be many different cuff design is an arrangement that will be suitable for use in the present invention.

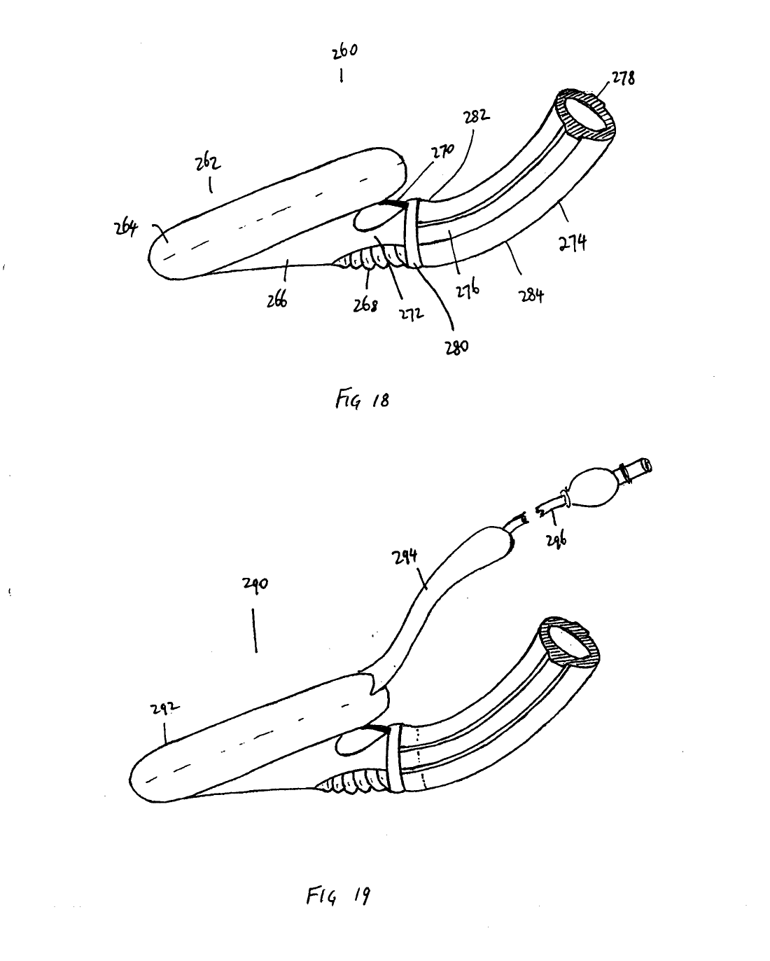

FIG. 18 shows a side view of a laryngeal mask 260 in accordance with another embodiment of the present invention. The laryngeal mask 260 includes a mask portion 262 having a flexible, conformable peripheral region 264 that, in use, can form a seal with the structures in the laryngopharynx of the patient. The laryngeal mask 260 includes a base plate 266. The base plate 266 has a corrugated region 268 and a thin membrane region 270. A region 272 of enhanced thickness or stiffness (when compared to the thickness or stiffness of regions 268, 270) extends between the regions 268 and 270.

The laryngeal mask 260 also has an airway tube 274. Airway tube 274 is connected to mask portion 262. Airway tube 274 includes thrust regions 276, 278 that, in use, are generally in alignment with region 272 (and a corresponding region on the other side of the base plate, not shown in FIG. 20) when the airway tube 274 is in its normal alignment. In this manner, a thrusting force applied to the airway tube can be passed along the region 272 to facilitate insertion of the mask into the patient.

The airway tube 274 is mounted to the base plate 266 of mask portion 262 about a swivel joint 280. By providing a swivel joint 280, the airway tube 274 can rotate relative to the mask portion 262. As can be seen from FIG. 20, the airway tube 274 has a generally straight short section 282 positioned close to the base plate 266 and a curved region 284 positioned proximally of the generally straight region 282. When the mask is inserted into a patient, if the head of the patient is rotated, the airway tube 274 will want to rotate with the head of the patient. If the airway tube 274 was rigidly connected to the base plate 266, rotation of the airway tube 274 would also cause the base plate 266 and the cuff 264 to rotate, thereby dislodging the cuff 264 from position in the patient's airway. This could lead to a potentially dangerous airway obstruction. However, as the airway tube 274 of the laryngeal mask 260 is connected to the base plate 266 by swivel joint 280, if the patient's head is rotated, the airway tube 274 can rotate relative to the cuff 264. As a result, rotation of the patient's head is much less likely to cause the cuff 264 to be dislodged from its correct position in the patient's airway.

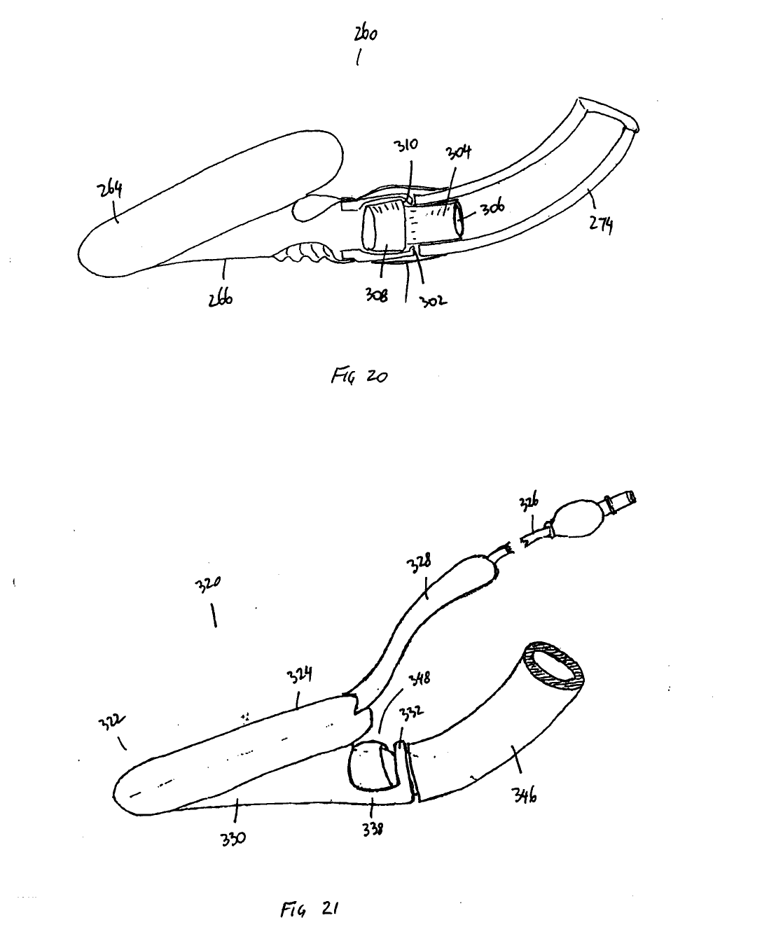

FIG. 19 shows a side view of a laryngeal mask 290 that has many similarities to the laryngeal mask 260 shown in FIG. 20. For convenience and brevity, the common features between FIGS. 18 and 19 will not be described in any further detail. However, the laryngeal mask 290 includes an inflatable cuff 292 that has a deformation means/tongue 294 and inflation tube 296. The deformation means/tongue may function as described in my international patent application number PCT/AU2010/000341.

FIG. 20 shows a partial cross sectional view of the mask 260 shown in FIG. 18. This figure has been provided to show details of how the airway tube 274 is connected to the base plate 266. In particular, base plate 266 may include a proximal opening that includes an inwardly extending flange 302. The distal end of the airway tube 274 has an adapter 304 glued therein. Adaptor 304 includes a central passageway 306 that extends completely through the adapter 304. A region of enlarged outer diameter 308 defines a shoulder 310 on the adapter 304. The opening of the base plate 266 is pushed over the region of the large diameter 310 until the inwardly extending flange 302 snaps over the shoulder 310 to thereby affix the base plate to the adapter. In this manner, the base plate 266 is firmly held to the adapter 304 so that the base plate 266 and cuff 264 cannot be removed from the airway tube 274 by application of a pulling force in the longitudinal direction. However, the adapter 304 can rotate relative to the base plate 266, thereby establishing a swivel joint between the base plate/mask portion of the laryngeal mask 260 and the airway tube 274.

FIG. 21 shows a further embodiment of the present invention in which a laryngeal mask is provided with a swivel joint between the mask portion and the airway tube. The laryngeal mask 320 shown in FIG. 21 includes a mask portion 322 having an inflatable cuff 324. Inflatable cuff 324 can be inflated via inflation tube 326. A deformation means/tongue 328 is affixed to a proximal end of the inflatable cuff 324.

The mask portion 322 also includes a base plate 330. Base plate 330 has a short tube region 332 (also see FIG. 24) that defines an opening 334. The proximal part of short tube region 332 is positioned proximally of a gap 336. Gap 336 is bounded by a dorsal wall portion 338, distal edge 340 and proximal edge 342. Dorsal wall portion 338 functions to provide elastic recoil to cause the mask to resume the natural or neutral position of the mask when the 328 is released after pulling to flex the joint. In other words, dorsal wall portion 338 functions as an elastic spring.

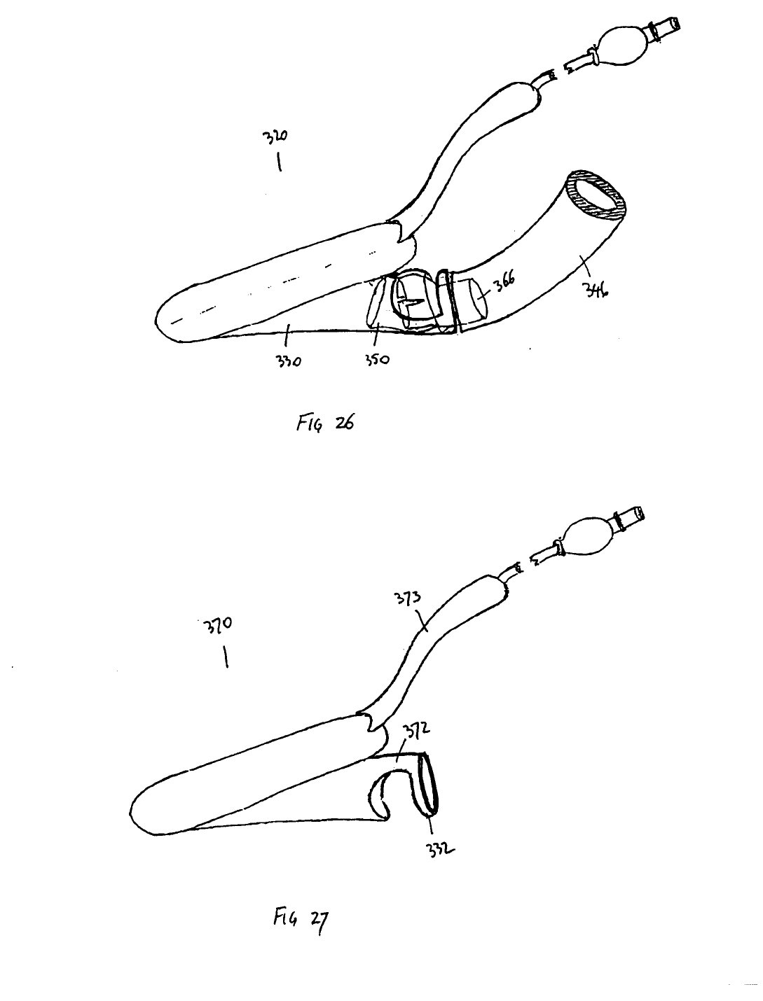

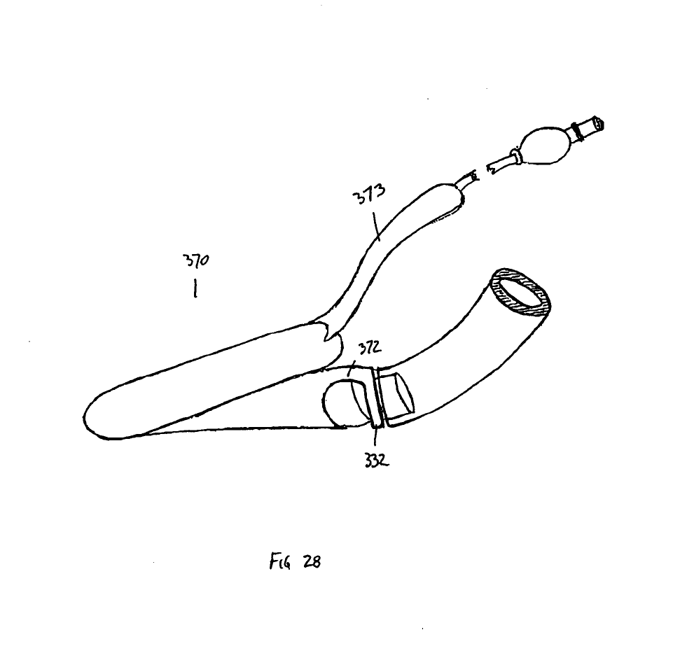

The laryngeal mask 320 shown in FIG. 21 also includes an airway tube 346. Airway tube 346 is connected to the base plate 330/mask portion 332 by a swivel joint shown schematically in FIG. 23 at 348. Swivel joint 348 is formed as a ball and socket joint. The components of the ball and socket joint are shown in FIGS. 23 and 24 and the joint components are shown in assembled form in FIG. 25.

The ball and socket joint comprises a socket portion 350 (see FIG. 23) and a ball portion 352 (see FIG. 24). The socket portion 350 includes a passageway extending therethrough. The socket portion 350 includes a distal part 354 that, in use, is inserted into the base plate past the distal edge 340. The distal part 354 may be glued or otherwise affixed to the inner walls of the passageway extending through the base plate 330. The socket portion 350 includes a rounded portion 356 having an opening 358 therein.

The ball portion 352 includes a rounded portion 360. The rounded portion 360 includes one or two cut-outs 362. In order to assemble the joint, the end 366 is inserted through opening 354 of the socket portion 350 until the position shown in FIG. 25 is reached. Due to the relative sizes of the rounded portion 360 of the ball portion and the rounded region 356 of the socket portion, the assembled ball and socket joint shown in FIG. 25 cannot be pulled apart by application of a longitudinal pulling force. In this manner, the joint does not come apart when traction on the airway tube is applied. The ball portion 352 includes a passageway 366 that extends therethrough. The distal end of the ball portion 352 will typically be joined to the airway tube 346, for example, by gluing or by plastics welding or ultrasonic welding.

As shown in FIG. 25, the ball and socket joint 348 allows relative movement between the base plate 330 and the airway tube 346 by allowing movement due to rotation and movement due to flexion or extension. The ball portion 342 may include a stop member 368 to limit movement caused by rotation or flexion/extension. The stop member 368 may, for example, allow up to 90° rotation either side of a centre line.

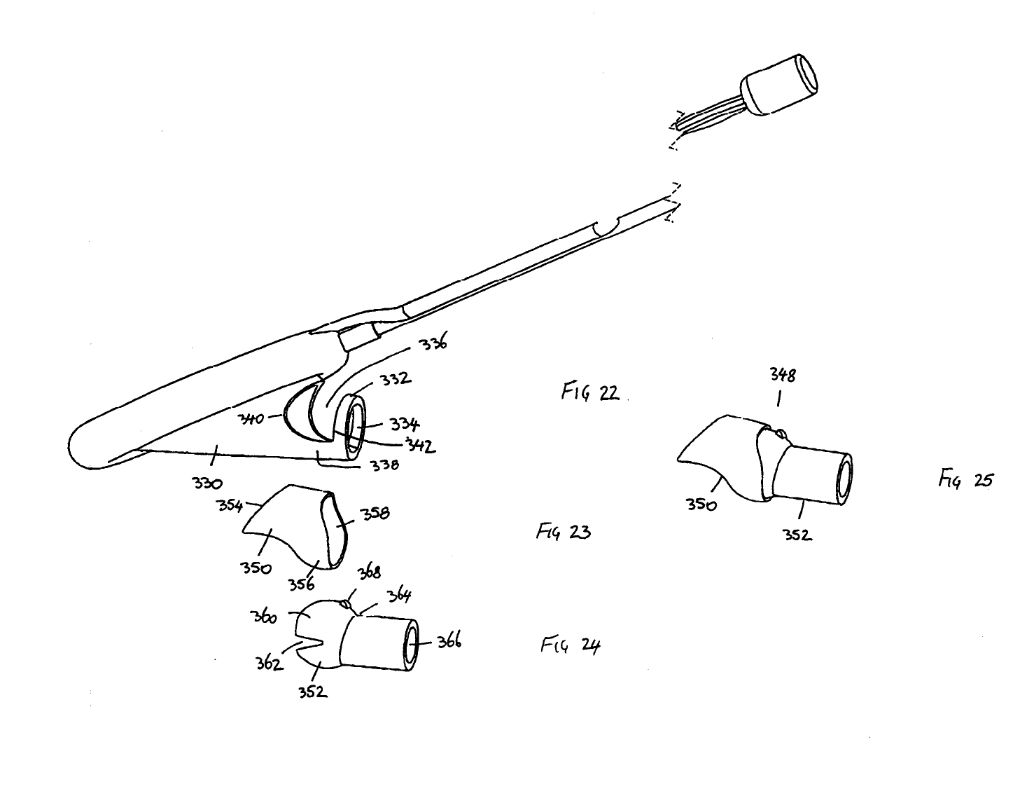

In the embodiment shown in FIGS. 21 to 25, the swivel joint not only allows the airway tube 346 to rotate relative to the base plate 330 in a direction around a longitudinal axis of the base plate, it also allows the airway 246 to move towards or away from the base plate by relative rotation around an axis extending laterally through the ball and socket joint. Thus, during insertion of the laryngeal mask 320 into the airway of the patient, the laryngeal mask 320 can “bend” around the joint 348 in order to enable the mask to adopt the required configuration to facilitate insertion of the mask past the back of the throat and into the airway passages of the patient. Once the mask has been properly inserted, if the head of the patient is rotated from side to side or if the head of the patient is extended backwards or forwards, relative rotation about the joint 348 is possible so that the mask portion 322 can remain correctly positioned in place in the laryngopharynx whilst the airway tube 346 moves with the movement of the head of the patient.

It will be appreciated that the ball and socket joint forms a universal joint that allows rotation about two separate axes. Furthermore, the ball and socket joint forms a part of the airway passage for providing ventilating gases or anaesthetic gases to the airway of the patient.

FIG. 26 shows the laryngeal mask 320 of FIG. 21 with details of the ball and socket joint being shown.

FIG. 27 and FIG. 28 show a variation of the laryngeal mask 320 shown in FIG. 21. The main difference between the laryngeal mask 370 shown in FIGS. 27 and 28, when compared to the laryngeal mask 320 shown in FIG. 21, is that the short opening 332 of the laryngeal mask 370 shown in FIGS. 27 and 28 is bounded by a ventral wall 372 (as opposed to a dorsal wall 338 as shown in FIG. 21). The function of ventral wall 372 is to act like a spring opposing the flexion created by pulling on the tongue 373. Ventral wall 372 acts to bring back the deformation of the mask in relation to the airway tube to its natural position. Both parts 372 and 338 may also contribute to the longitudinal thrust in addition to the thrust exerted through the hard ball and socket joint.

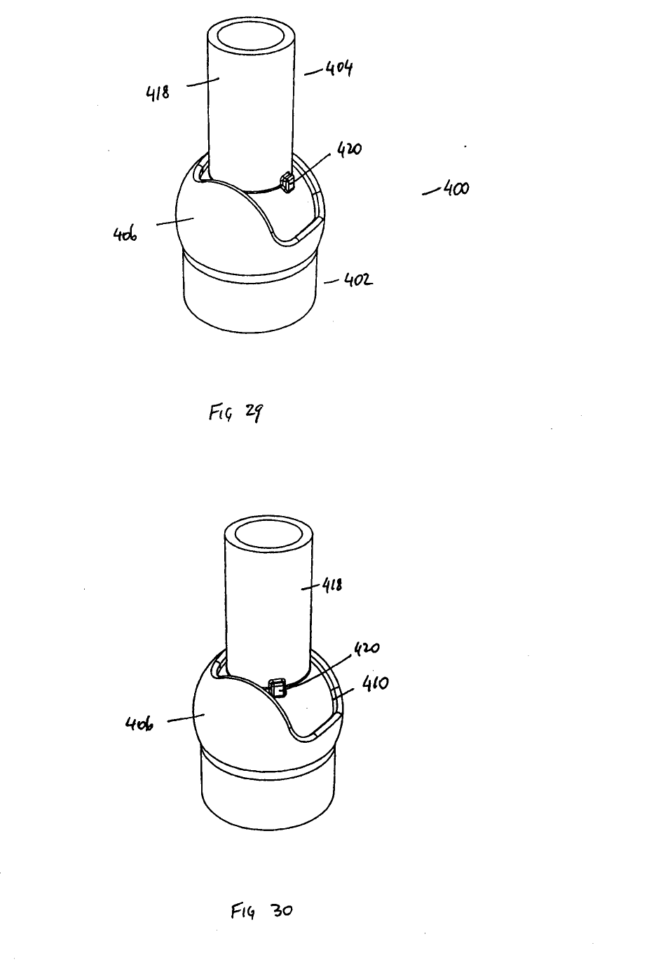

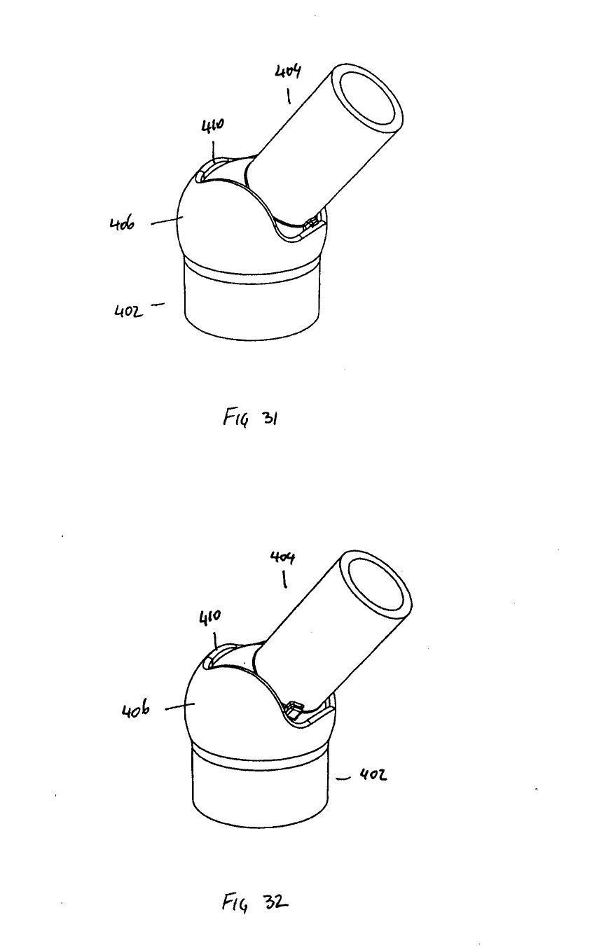

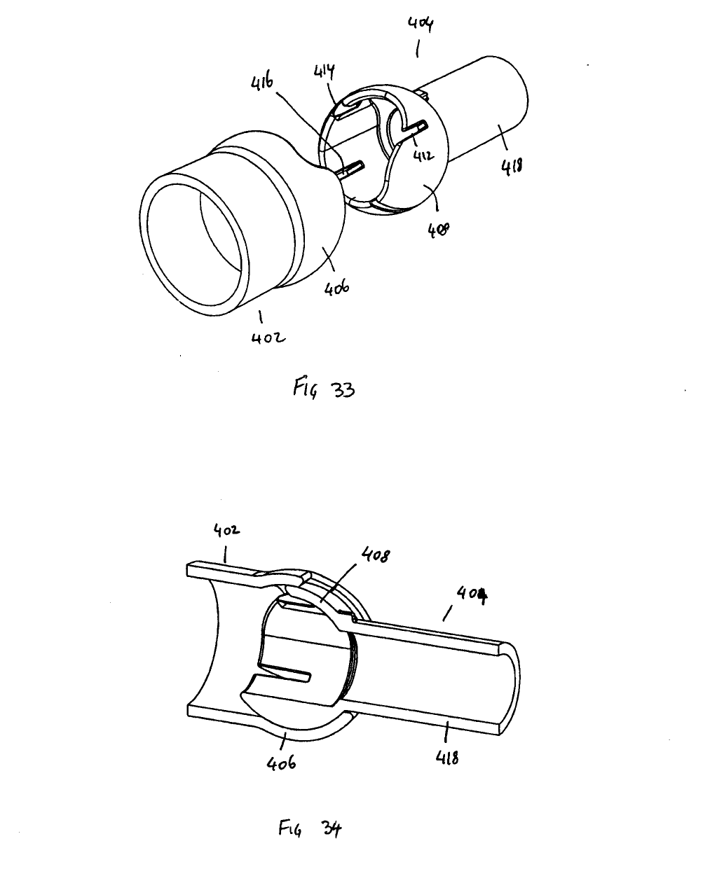

FIGS. 29 to 34 shown various views of an alternative swivel joint for use in embodiments of the present invention. In FIGS. 29 to 34, the swivel joint is in the form of a universal joint, formed by a ball and socket connection. The swivel joint 400 comprises a socket portion 402 and a ball portion 404. Socket portion 402 includes an enlarged round portion 406. Ball portion 404 includes an enlarged ball portion 408 that, in use, is received within enlarged round portion 406 of the socket portion 402. The enlarged round portion 406 of the socket portion 402 includes an opening 410. Ball portion 408 passes through opening 410 in order to connect the ball and socket portions together. Ball portion 408 may include spaced apertures 412, 414, 416 that enable the ball portion to effectively reduce in size as it is being inserted through the opening 410. Once the ball portion 408 has been fully inserted through the opening 410, it snaps outwardly to come into contact with the inner wall of the enlarged round portion 406 of the socket portion 402.

The ball portion 408 includes an extension 418 that defines a passageway. In use, the extension 418 is glued or otherwise affixed to the airway tube.

The ball portion 418 also includes a stop member 420. Stop member 420 is used to limit the relative rotation between the ball and socket joint.

FIGS. 29, 30, 31 and 32 show some of the degrees of rotation allowed by the ball and socket joint. In use, the ball portion is mounted to the airway tube and the socket portion is mounted to the mask portion/base plate. Of course, this mounting may be reversed.

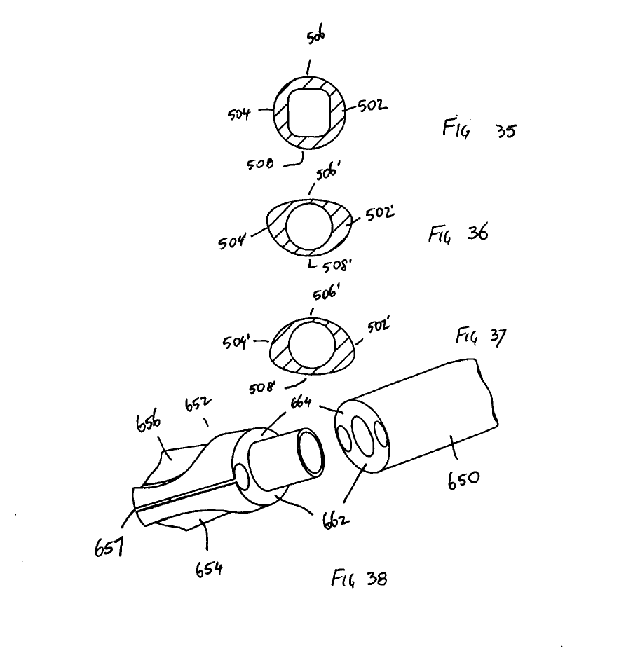

FIGS. 35, 36, and 37 show cross-sectional views of either the airway tube or the airway extension of the base plate of the laryngeal mask. In FIG. 35, the airway tube or the airway extension of the base plate has a generally circular outer periphery and a non-circular inner periphery such that the dorsal wall 506 and the ventral wall 508 at 11 in the lateral walls 502, 504. In FIG. 36, the airway tube or the airway extension of the base plate includes thick wall regions 502′, 504′ that separate a thin membrane region 506′ in the ventral part of the airway tube or base plate and a dorsal corrugated region 508′ in the airway tube or base plate. The dorsal corrugated region 508′ allows the laryngeal mask to bend in a manner that extends the corrugated region 508′ whilst, at the same time, effectively shortening the ventral region 506′. The airway tube or the airway extension of the base plate shown in FIG. 38 has a generally circular cross section for the inner periphery of the flow passage. The outer periphery is of an oval cross-section or other non-circular cross-section.

FIG. 37 shows a similar cross-sectional view, but with a slightly different cross-sectional shape. For convenience and brevity, the regions in FIG. 38 that are similar to the regions in FIG. 37 are denoted by the same reference numerals.

FIG. 38 shows a schematic view of how an extruded triple lumen airway tube 650 can be connected to the mask portion/base plate portion 652 of the laryngeal mask. In particular, the mask portion/base plate portion 652 has a flexible corrugated region 654 and a flexible thin wall region 656. A male fitting 658 is connected to or formed with an airway passageway in the mask portion/base plate portion 652. This male fitting 658 may be relatively rigid. The male fitting 658 is inserted into the airway passage 660 of the triple lumen tube 650 and glued into place to hold the portions together. The relatively thick wall portions, denoted by reference numerals 662 and 664, allow a thrusting force to be transmitted therealong. Grooves or tubes formed in the mask portion (one of which is shown at 657) come into alignment with the distal outlets of the outer tubes of the triple lumen tube 650.

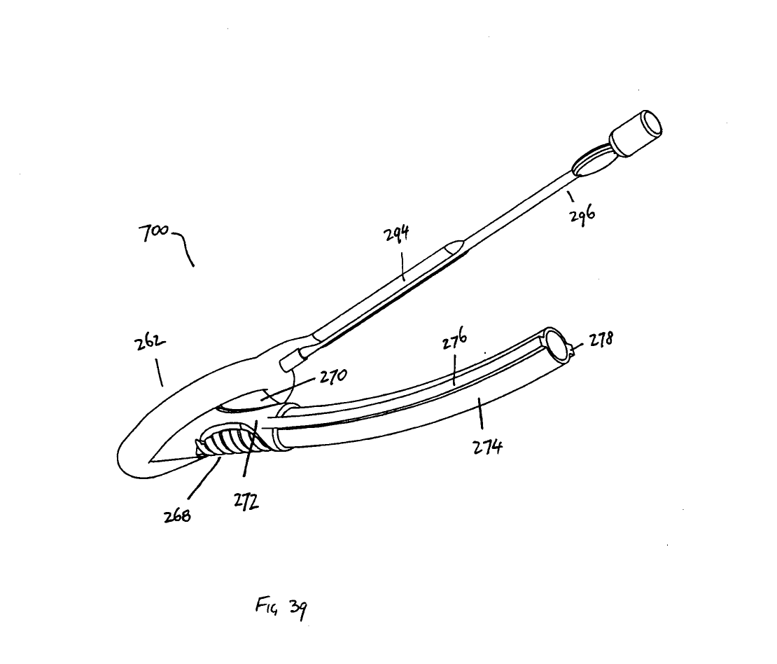

FIG. 39 shows a perspective view of a laryngeal mask 700 that is similar to the laryngeal mask shown in FIG. 19, except that the mask portion 262 and the airway tube 274 are integrally formed. The other features of the mask 700 are similar to the features as shown in FIGS. 18 and 19 and, for convenience and brevity of description, the same reference numerals have been used to denote those features in FIG. 39.

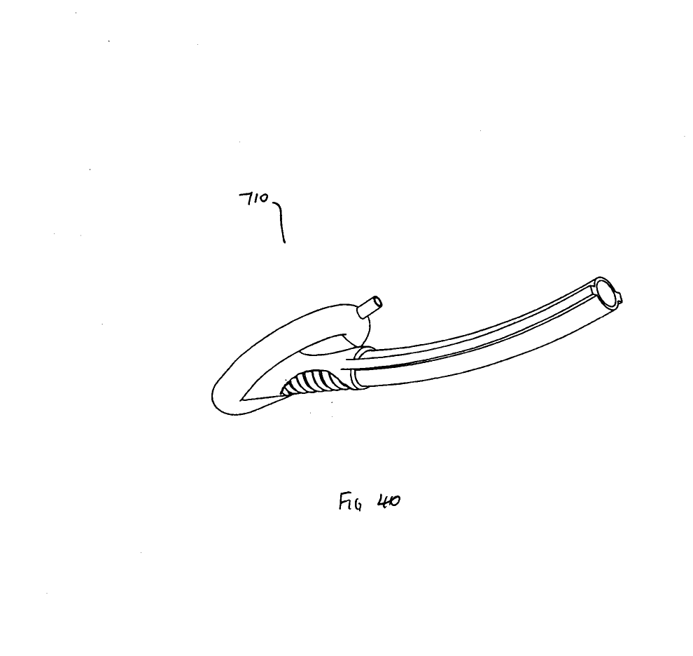

FIG. 40 shows a perspective view of a laryngeal mask 710 that is similar to the laryngeal mask shown in FIG. 18, except that the mask portion 262 and the airway tube 274 are integrally formed. The other features of the mask 710 are similar to the features as shown in FIGS. 18 and 19 and, for convenience and brevity of description, the same reference numerals have been used to denote those features in FIG. 40.

FIG. 41 shows a side view of a laryngeal mask 720 in accordance with another embodiment of the present invention. The laryngeal mask 720 includes a mask portion 722 and an airway tube 724 connected thereto. The mask portion 722 has an inflatable cuff 726 that is able to be selectively inflated and deflated via inflation tube 728. The mask portion also includes a base plate 730. The base plate 730 has a corrugated dorsal region 732 and a corrugated ventral region 734. A region of stiffer material 736 extends between the corrugated region 732 and 734. It will be appreciated that the stiffer region 736 may be made from the same material as the corrugated regions 732, 734, but with the region 736 being stiffer by virtue of the fact that it does not have a corrugated configuration. It will also be understood that the stiffer region 736 may have the same wall thickness of the wall thickness of the corrugated regions 732, 734. Again, the stiffer region 736 is stiffened by virtue of the different configuration of the side wall when compared to the configuration of the side wall of the more flexible regions.

In order to connect the mask portion 722 to the airway tube 724, a connector 738 is glued or otherwise joined to the distal end of the airway tube 724. The connector 738 includes a first region that fits within the internal diameter of the airway tube 724 and a second region 740 of enlarged outer diameter. This forms a shoulder 742 that engages with a corresponding shoulder 744 that is formed in the inner surface of an airway extension of the base plate 730. In order to connect the airway tube 724 to the base plate 722, the region of enlarged outer diameter 740 is pushed into the opening of the airway extension of the base plate 730 until the shoulder 742 passes beyond the shoulder 744. This causes the shoulder 744 to snap back onto and engage with the shoulder 742 to thereby retain the connector within the airway extension of the base plate 730.

As can be seen from FIG. 41, the part of the connector 738 that extends into the airway tube 724 extends at a different angle to the part of the connector 738 that extends into the airway extension of base plate 730. This is shown by dashed line 746, which represents the longitudinal axis of the part of the connector 738 that extends into the airway tube 724, and dashed line 748, which represents the longitudinal axis of the part of the connector 738 that extends into the airway extension of base plate 730. In particular, axis 748 extends distally and ventrally of axis 746. The present inventor has found that positioning the mask portion 730 so that it effectively has a line on a dorsal-most outer surface that extends from a proximal part to a distal part and extends in a ventral direction facilitates insertion of the mask into the airway of the patient.

The mask 720 of FIG. 41 also includes a cover 750 is positioned over the connection between the mask portion 722 and the airway tube 724. The cover may be glued to the laryngeal mask 720. Alternatively, the cover may be fully formed with the mask portion of the airway tube and subsequently glued or otherwise affixed to the other part during assembly. The cover and assists in minimising or stopping any leaks from the connection between the mask portion 722 and the airway tube 724. The cover also assists in stopping any penetration of liquids or fluids through the connection from the outside of the mask.

FIG. 42 shows a dorsal view of a laryngeal mask 770 in accordance with another embodiment of the present invention. FIG. 43 shows a perspective view from the ventral side of the mask 770 shown in FIG. 42. The mask 770 includes a mask portion 772 and an airway tube 744. The mask portion 772 includes an inflatable cuff 776. The inflatable cuff 776 is of a generally annular or near round shape for easy insertion and positioning. The mask portion 772 also includes a dorsal corrugated region 778 and a ventral corrugated region 780. Although not clearly shown in FIGS. 42 and 43, relatively stiff regions extend between and to the proximal and distal sides of corrugated regions 778, 780.

The mask 770 is also provided with tubes 782, 784 that have respective distal outlets 786, 788. These tubes may be provided for removing vomitus or other gastric juices that may rise up from the oesophagus during use of the laryngeal mask. The mask also includes a projection 790 located at the distal end of the mask portion 772. The projection 790 assists in preventing the distal opening 786, 788 of tubes 782, 74 from becoming blocked by tissues of the patient being sucked into the openings. The projection 790 also assists in holding open the oesophagus to at least a small extent.

The mask 770 also includes a tongue 792 attached to a ventral portion of the mask portion 772. The tongue 792 enables the mask to be selectively deformed by pulling on the tongue during insertion. An inflation tube 794 is also provided for selectively inflating and deflating the inflatable cuff 776.

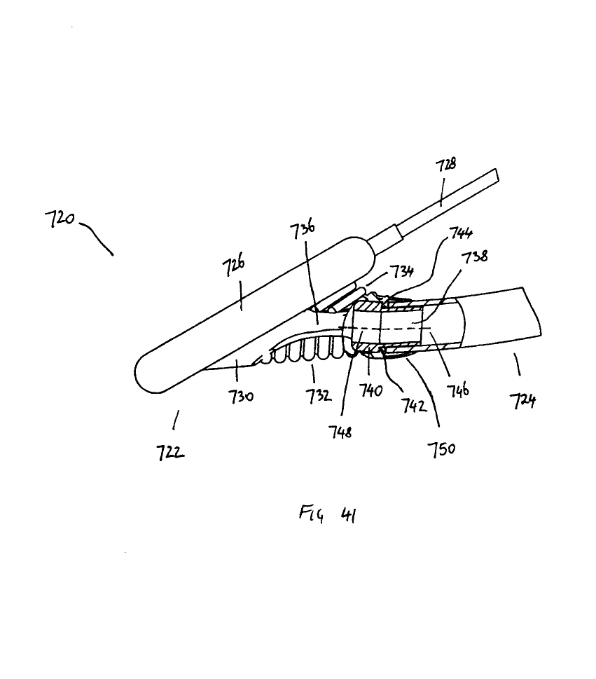

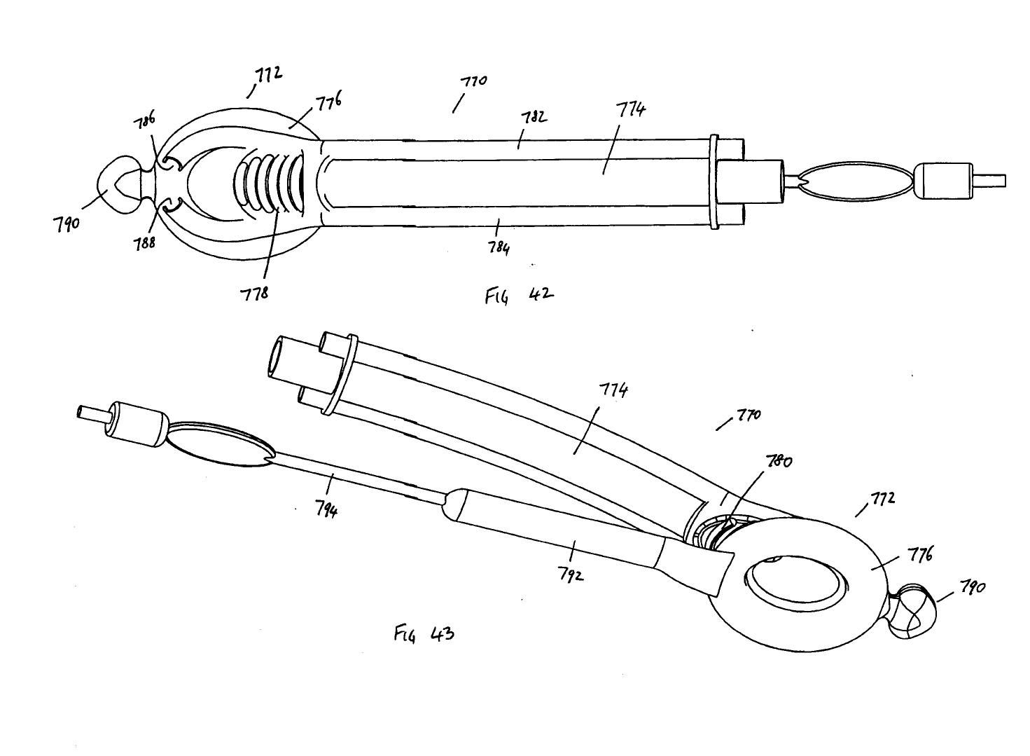

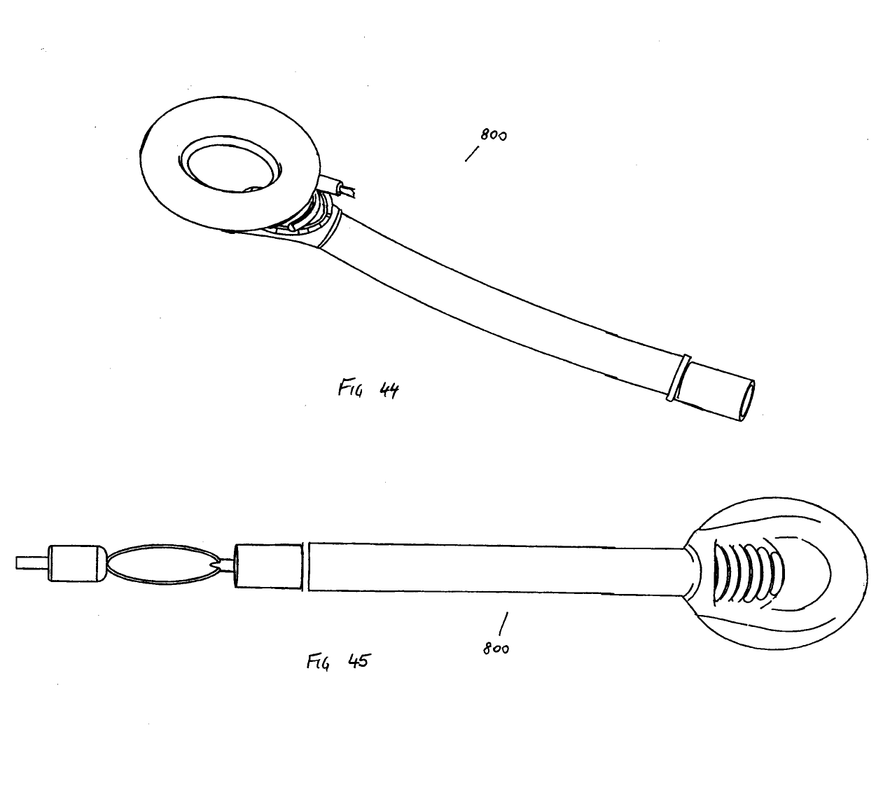

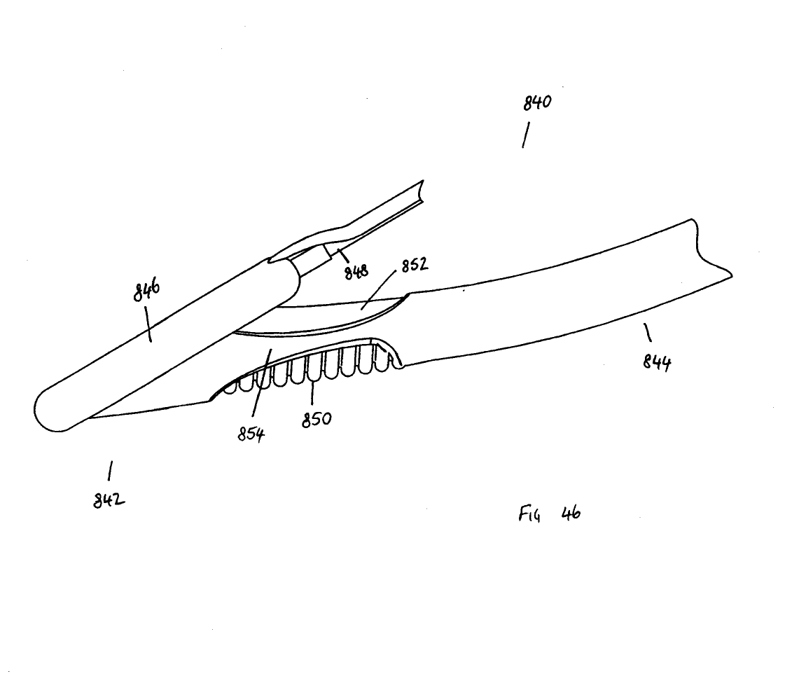

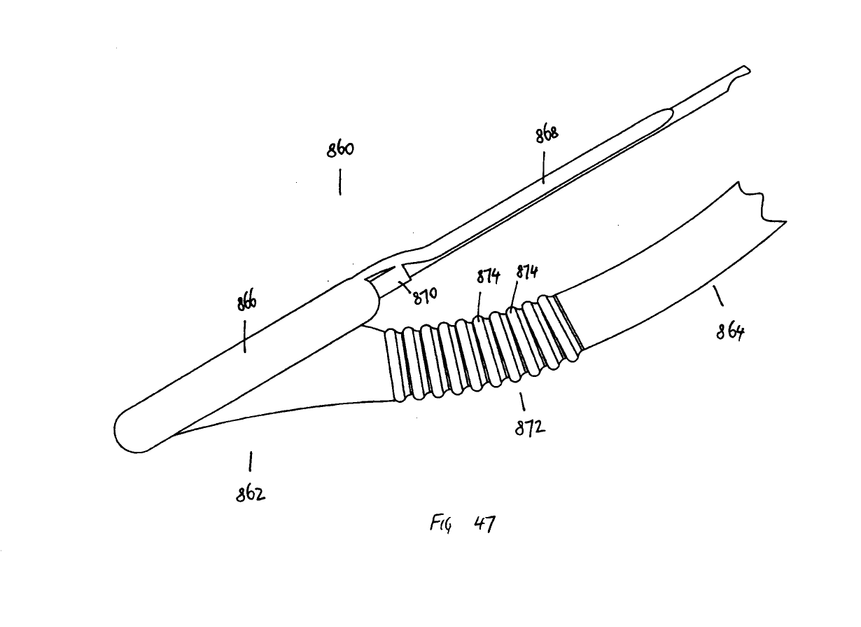

FIGS. 44 and 45 show a perspective view from a ventral side and a dorsal view, respectively, of a laryngeal mask 800 in accordance with yet another embodiment of the present invention. This laryngeal mask is generally similar to the laryngeal mask 770 shown in FIGS. 44 and 45, except that the distal projection 790 and the tongue 792 have been omitted from laryngeal mask 800. Other features of the mask are similar to those as shown in FIGS. 43 and 44 and have not be described further.

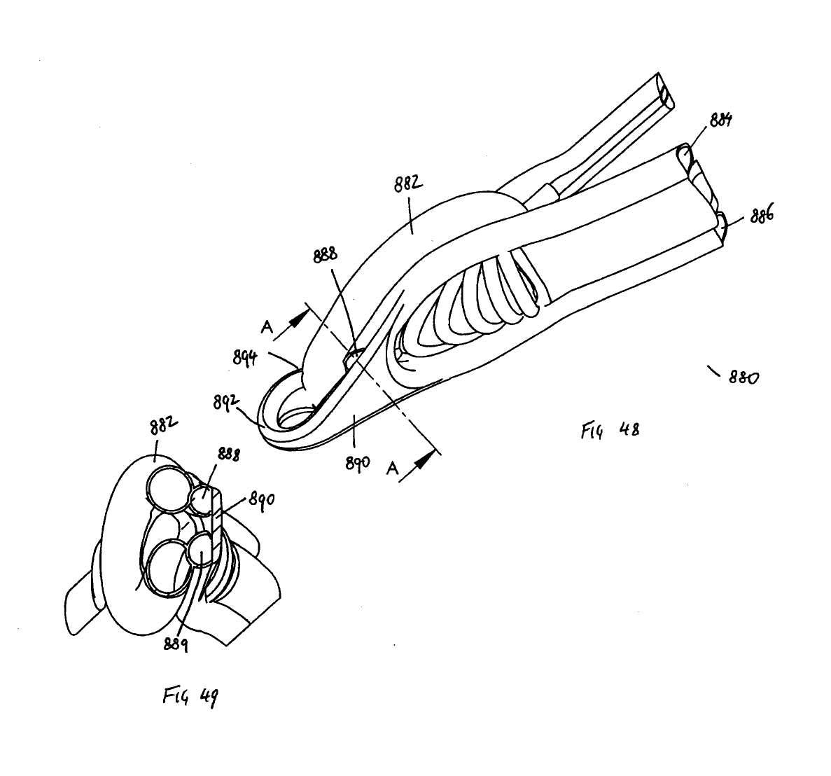

FIG. 46 shows a side view of a laryngeal mask 840 in accordance with a further embodiment of the present invention. The laryngeal mask 840 includes a mask portion 842 and an airway tube 844. The mask portion 842 has an inflatable cuff 846 that can be selectively inflated and deflated by inflation tube 848.

The mask 840 includes a corrugated dorsal region 850 located at a proximal part of the mask portion 842. The mask portion 842 also includes a ventral region 852 that is covered by a thin membrane. Ventral region 852 has enhanced flexibility when compared with the region 854 that extends between the corrugated region 850 and region 852.

The mask 840 is integrally formed. The airway tube is considered to have its distal region located proximally of the regions 850, 852. In this regard, the mask portion 842 will typically include an airway extension in which the configuration of the airway extension changes from the chamber part of the mask portion 842 into the consistent cross-section of the airway tube 844. The mask portion 842 may be considered to have its proximal region located at a region where the configuration of the airway extension becomes essentially constant.

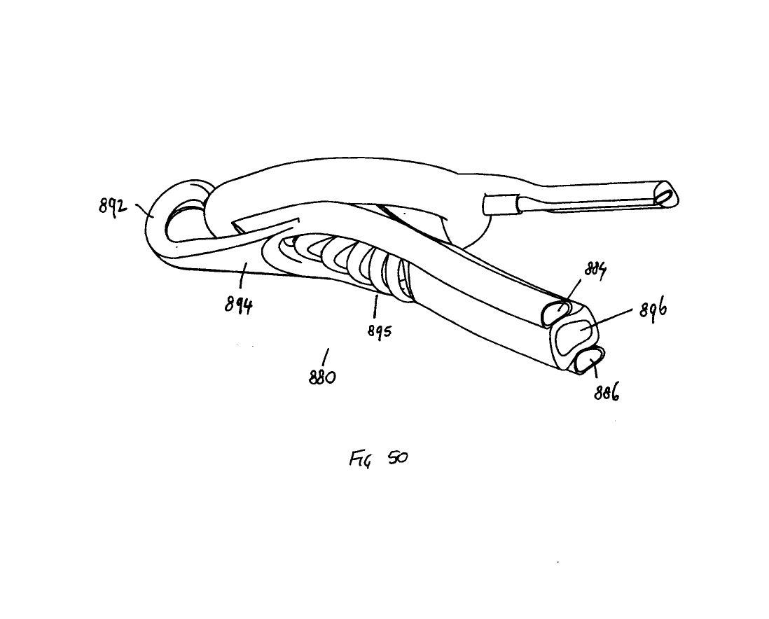

FIG. 47 shows a side view of a laryngeal mask 860 in accordance with a further embodiment of the present invention. The mask 860 includes a mask portion 862 and an airway tube 864. The mask portion has an inflatable cuff 866, a tongue 868 and an inflation tube 870.

The airway tube 864 has its distal end formed by a corrugated region 872. The corrugated region 872 may have a wall thickness that is essentially the same as the wall thickness of the proximal end of airway tube 864. The corrugated region 872 provides a region of enhanced flexibility in the airway tube. However, thrust can also be transmitted through the corrugated region 872 during insertion of the mask 860 into the airway of the patient. In this regard, applying a thrust to corrugated region 872 could result in the corrugations collapsing upon themselves so that the thicker ridges (some of which are numbered at 874) come into contact with each other and effectively form a continuous line of stiffer material. Alternatively, if the corrugated region 872 becomes sufficiently bent or angled, the part of the ridges 874 at the inner part of the bend or angle will also come into contact with each other and therefore form an effective continuous line of material that can transmit a force.

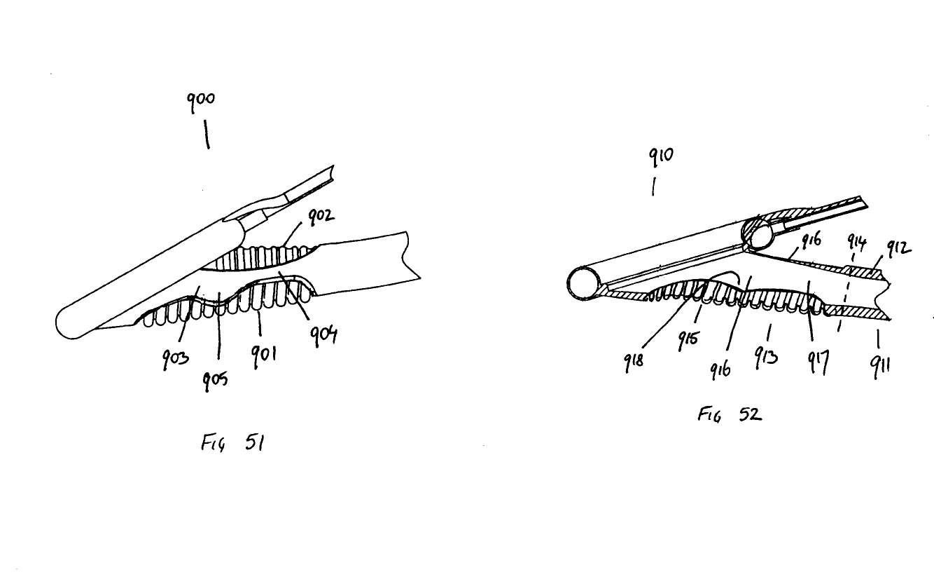

FIG. 48 shows a perspective view from a dorsal side of the laryngeal mask 880. FIG. 49 shows a perspective view looking from a distal end of the mask 880 and showing a cross-section taken along line A-A of FIG. 48. The mask 880 shown in FIGS. 48 and 49 has a number of features in common with the mask 810 shown in FIG. 13 and, for reasons of brevity of description, similar features will not be described further.

The mask 880 includes an inflatable cuff 882 and gastric drainage tubes/gastric vent tubes 884, 886. Distal opening 888 of tube 884 is shown in FIG. 48. A projection 890 extends from the dorsal part of the mask portion. As can be seen from FIGS. 52 and 53, the proximal part of projection 890 is of sufficient lateral width to extend across the top of the distal openings 888, 889 of the gastric tubes 884, 886. The projection 890 extends into a loop 892 that joins to a ventral part of the cuff at 894. The loop 892 assists in pushing away the tissues of the oesophagus and maintaining those tissues away from the distal openings 888, 889 of tubes 884, 885, was also assisting in maintaining the oesophagus at least partly open.

FIG. 50 is a further view of the laryngeal mask 880 shown in FIGS. 48 and 49, but showing a larger degree of flexing or bending in the flexible region 895 of the mask portion. Also shown in FIG. 50 is the cross sectional shape of the airway tube 896, which may not necessarily be of cylindrical cross section. Indeed, the inner passageway of airway tube 896 shown in FIG. 50 is somewhat elongated in a dorsal to ventral direction, when compared to the lateral width thereof.

FIG. 51 shows a side view of a laryngeal mask 900 in accordance with another embodiment of the present invention. The laryngeal mask 900 includes a dorsal corrugated region 901 and a ventral corrugated region 902 in the mask portion/baseplate. A region of stiffer material 903 extends between the corrugated region's 901, 902 (and a similar region of stiffer material is positioned on the other side of the mask). The region 903 includes a relatively narrow portion 904 and a relatively wider portion 905. Bending in the vicinity of relatively narrow portion 904 will be promoted. Thrust can be applied along the region 903 of stiffer material. It will also be understood that the region of stiffer material may have a similar wall thickness and be made from the same material as the corrugated regions 901, 902 but be stiffer because it does not have the corrugated configuration.

FIG. 52 shows a cross sectional side view of a laryngeal mask 910 in accordance with a further embodiment of the present invention. As can be seen, the airway tube 911 has a relatively constant wall thickness 912. The mask portion 913, which commences at around dashed line 913, has a ventral corrugated region 915 and a dorsal region 916 of reduced wall thickness. A stiffer region 916 that has a wall thickness that is generally the same as wall thickness 912 extends between regions 915 and 916. Stiffer region 916 also extends proximally and distally of the regions 915, 916. The stiffer region 916 also includes a narrow part 917 and a wider part 918. This promotes bending or flexing around the narrow regions thereof.

FIG. 53 shows a side view of a laryngeal mask 920 in accordance with another embodiment of the present invention. The mask 920 has a number of features that are similar to the mask 900 shown in FIG. 51 and for brevity of description similar features will be denoted by similar reference numerals but with a ′ added. The main difference between mask 900 shown in FIG. 51 and the mask 920 shown in FIG. 53 is that the region of relatively stiffer material or region that is considerably less amenable to bending 903′ has a slightly different shape to the region 903 shown in FIG. 51.

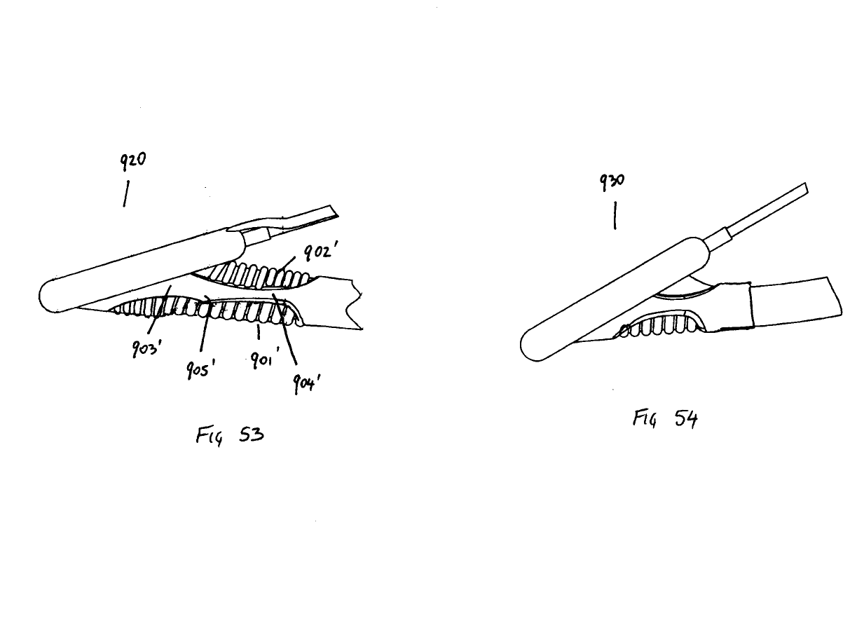

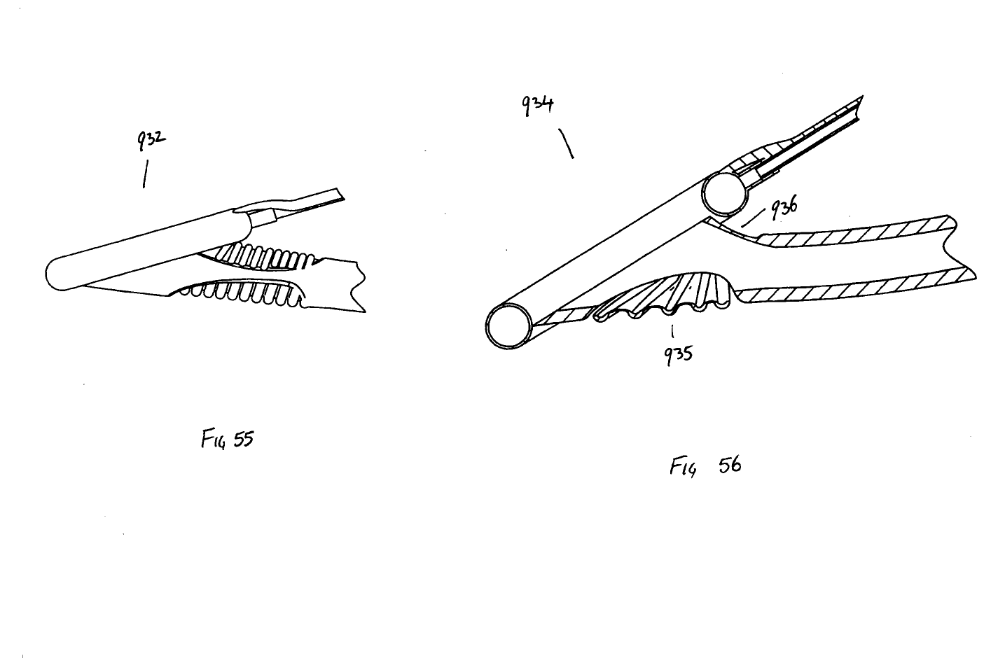

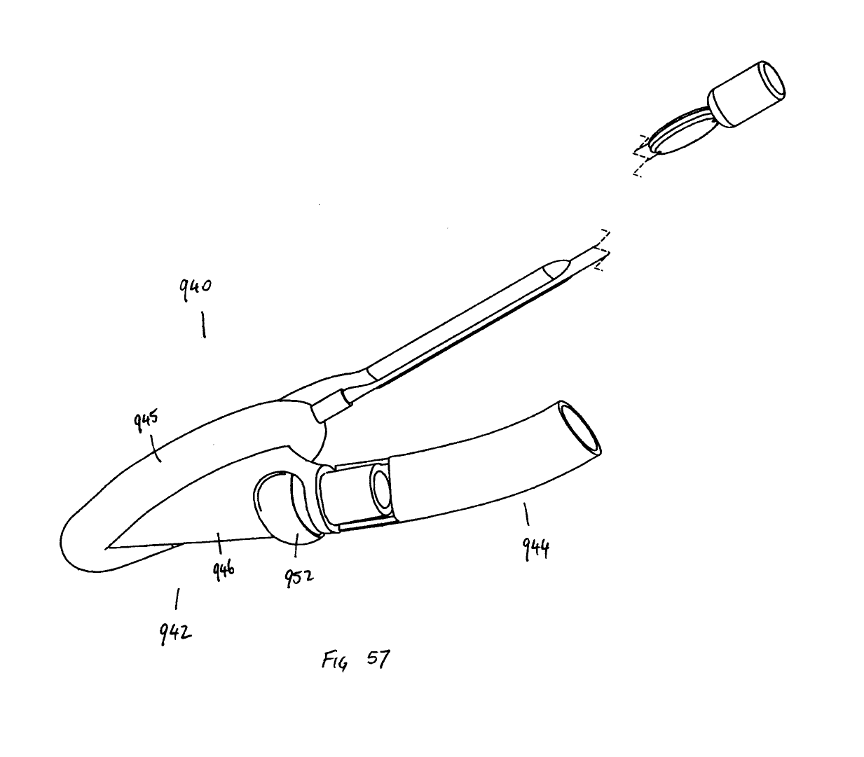

FIGS. 54 and 55 show laryngeal masks 930, 932 that have a number of similarities to the masks showing in FIGS. 56 to 58, but with slightly different arrangements of features thereof.

FIG. 56 shows a laryngeal mask 934 that has a number of features in common with the mask 910 shown in FIG. 35, but with slightly different arrangement of features. In particular the corrugated region 935 and the thin walled region 936 have a significantly less length in the proximal to distal direction than the equivalent features in FIG. 35.

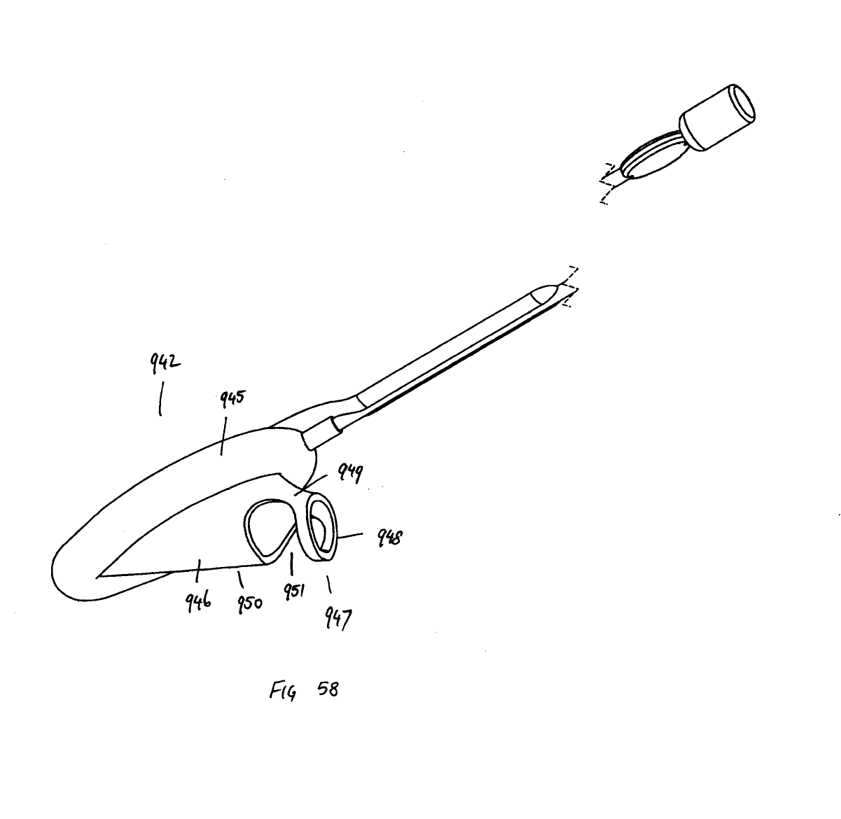

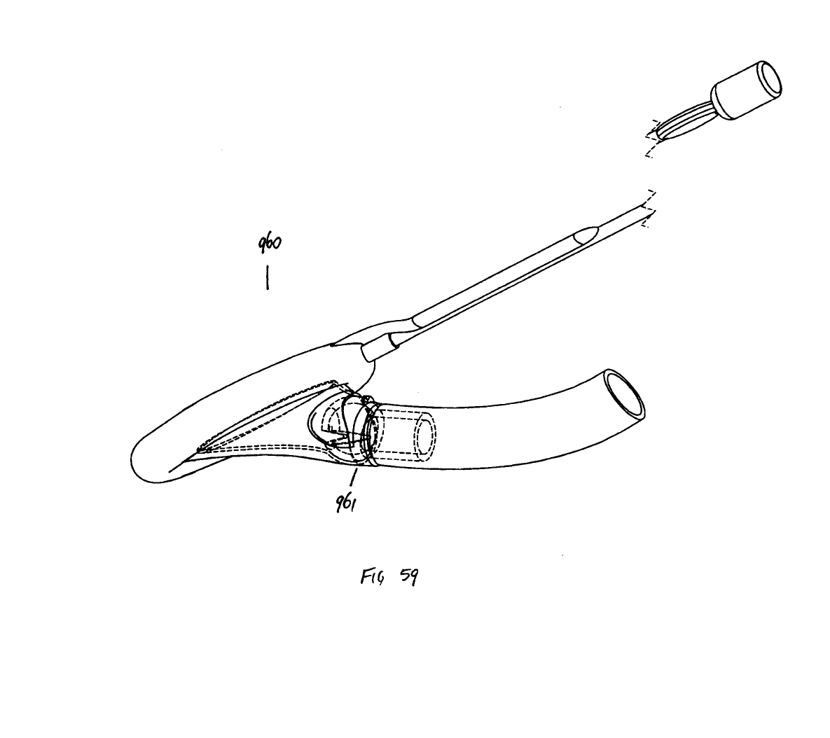

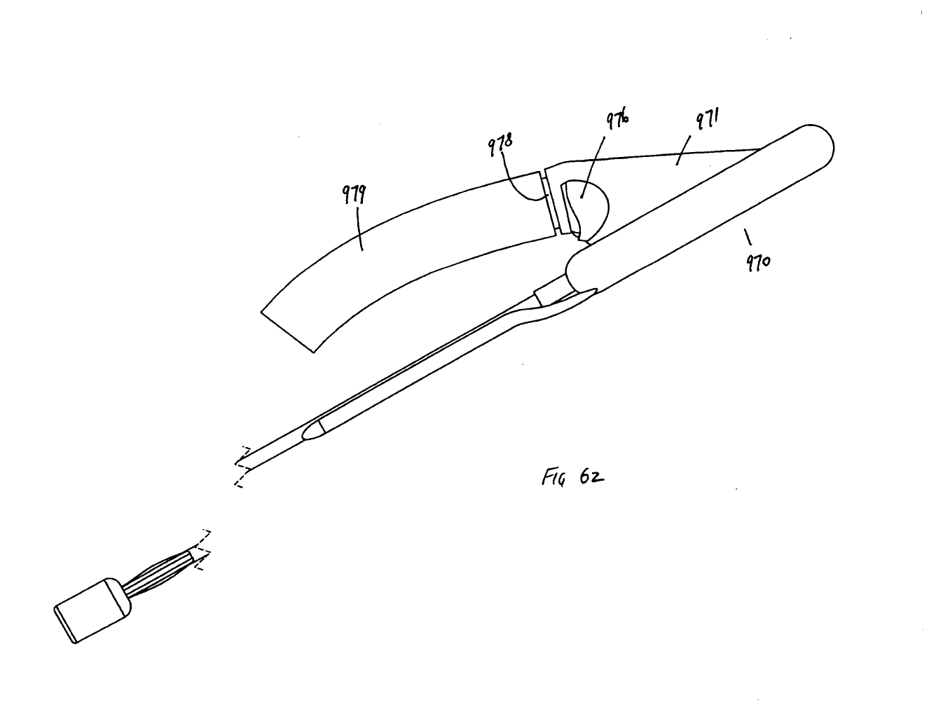

FIGS. 57 and 58 shown perspective views of another embodiment of a laryngeal mask in accordance with the present invention. FIG. 57 shows the completed mask with the airway tube attached (part of the airway tube is shown in cross-section) whilst FIG. 58 shows the mask portion with the airway tube not attached. The laryngeal mask 940 shown in FIG. 62 includes a mask portion 942 and an airway tube 944. The mask portion 942 includes an inflatable cuff 945 and a base plate 946. The base plate 946 defines a connecting region 947. The connecting region 947 has a proximal end of a part 148 and is joined by a ventral wall 949 to the main part 950 of the baseplate 946. A gap or space 951 is located dorsally of ventral wall 949. A joint 952, which may be similar to the connection joint shown in FIG. 25, or as shown in FIGS. 29 to 34, is used to connect the airway tube 944 to the mask portion 942. The ventral wall 949 allows thrust that is applied to the airway tube 944 to be transferred to the mask portion 942 during insertion of a laryngeal mask 940. Furthermore, the mask portion 942 can flex about the ventral wall 949 to enable the mask portion to bend or flex as required during insertion into the patient's airway.

FIG. 59 shows a view of another laryngeal mask 960. The mask 960 is similar to mask 940 shown in FIG. 57. However, instead of having a ventral wall 949, mask 960 has a dorsal wall 961. The connector shown in FIG. 64 is the same as the connector shown in FIGS. 29 to 34.

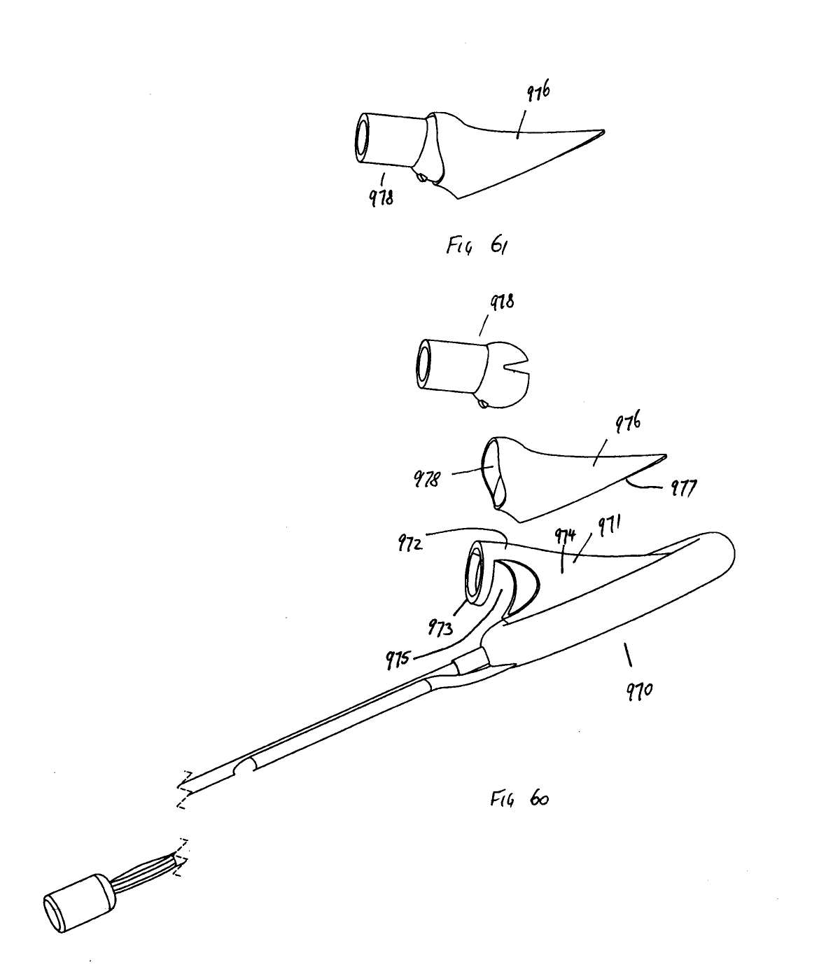

FIG. 60 shows a mask portion 970 that has a similar back plate arrangement 971 as that shown in FIG. 59. This back plate arrangement 971 includes a ventral wall 972 that connects annular proximal region 973 to the main portion 974 of the back plate. A space 975 is defined between the annular region 974 and the main portion 974 of the back plate 971.

In the embodiment shown in FIG. 60, the back plate 971 may be made from a soft and flexible material. In order to provide additional strength to the back plate 971, a back plate insert 976 may be provided. Back plate insert 976 has a ventral opening 977 and a proximal opening 978. The back plate insert 976 is inserted into the chamber defined by the main portion 974 of the back plate 971. The back plate insert 976 may be glued into the chamber. A connector 978, which may be the same as the connector shown in FIGS. 31 to 36, is connected to the back plate insert 976 in the manner as shown in FIG. 66. An airway tube may subsequently be connected to the proximal end of connector 978.

FIG. 61 shows an airway tube 979 being connected to the mask portion 970.

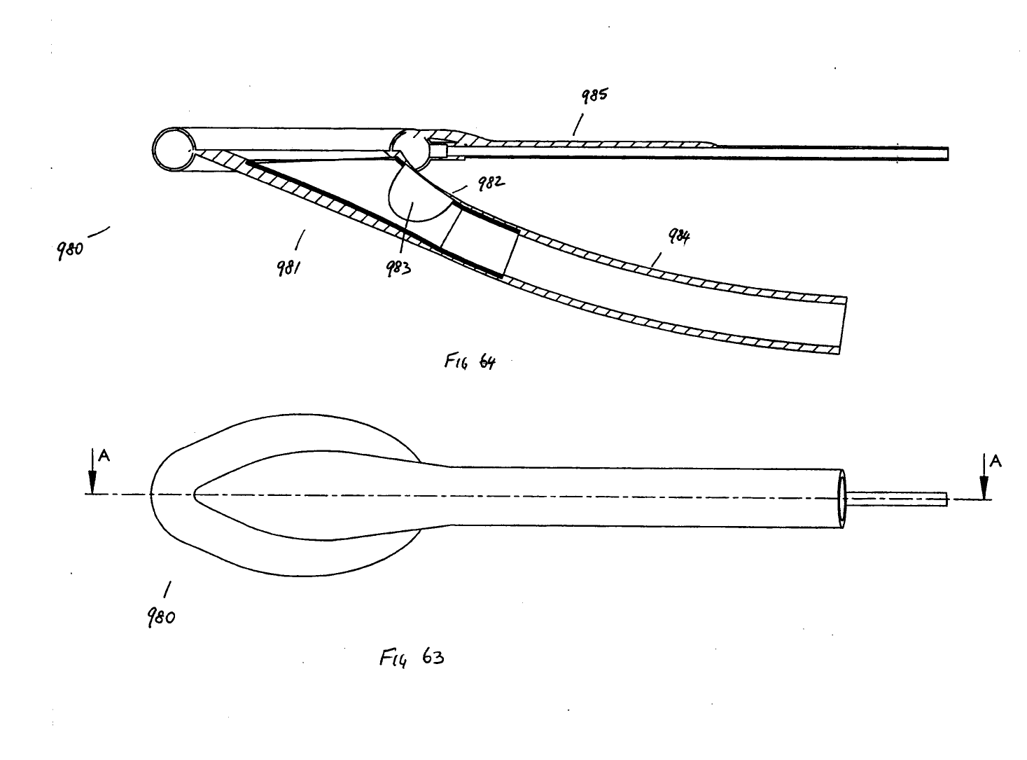

FIG. 63 shows a ventral view of a laryngeal mask 980. FIG. 64 shows a cross-sectional view taken along line A-A of FIG. 63. The mask portion 981 of the laryngeal mask 980 is generally similar to that as shown in FIG. 21. A cover 982 extends over the space 983 and the joint where the airway tube 984 is connected to the mask portion 981 to prevent ingress or egress of fluids through the space and the joint. The thicknesses of the various wall sections are also shown in FIG. 64.

The mask portion 981 also includes a deformation tongue 985 to facilitate controlling of the shape of the laryngeal mask 980 during insertion. This tongue may be as described in my international patent application number PCT/AU2010/000341, the entire contents of which are herein incorporated by cross-reference. In some embodiments, the tongue may be connected to the mask portion by a septum or by a web of material.

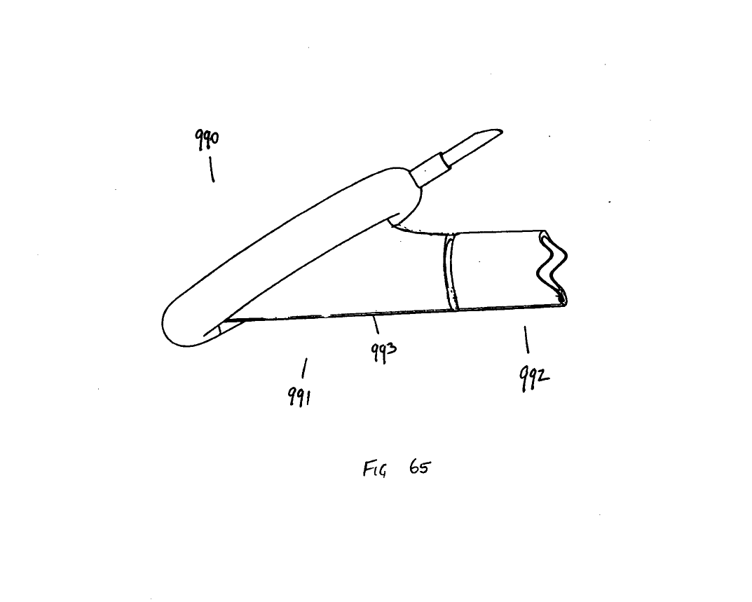

FIG. 65 shows a laryngeal mask 990 in accordance with another aspect of the present invention. The laryngeal mask 990 shown in FIG. 65 has a mask portion 991 connected to an airway tube 992. Unlike the previously described embodiments of the present invention, mask 991 does not include regions of increased flexibility or reduced wall thickness. Instead, the mask 990 is arranged such that a dorsal most line 993 extending from a proximal location to a distal location of the mask portion 991 is generally parallel to the longitudinal axis of the distal end of the airway tube 992.

FIG. 66 shows a mask 995 that is similar to the mask 990 shown in FIG. 70, except that the dorsal most line 996 of the mask portion 997 extends at an angle to the longitudinal axis of the distal end of the airway tube 998. Line 996 extends at an angle to the longitudinal axis of the distal end of the airway tube 998 such that the distal end of line 996 is located ventrally of the proximal end of line 996.

The present inventor has surprisingly found that providing a laryngeal mask as shown in FIG. 65 or 66 facilitates or enhances insertion and positioning of the mask without necessarily requiring the flexible and stiffer regions of other embodiments of the present invention.

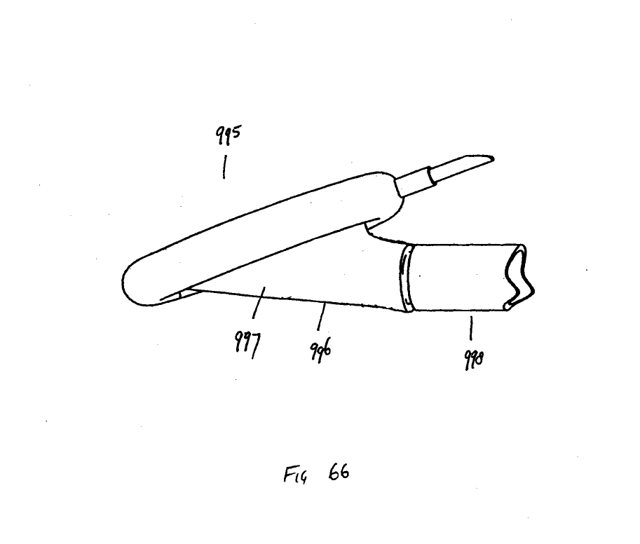

FIG. 67 shows a laryngeal mask 1000 that has the same angular arrangement of the dorsal most line of the mask portion 1001 and the longitudinal axis of the distal end of airway tube 1002 as shown in FIG. 66, but with a connection joint 1003 being used to connect the airway tube to the mask portion. FIG. 68 shows a similar mask 1005 having the straight arrangement between the dorsal most line of the mask portion 1006 and the longitudinal axis of the distal end of the airway tube 1007.

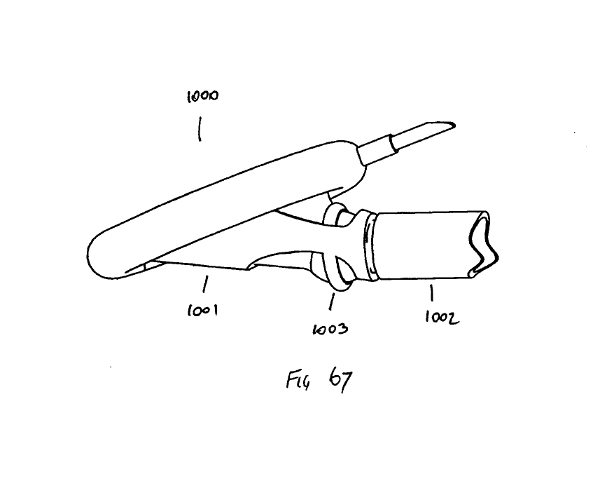

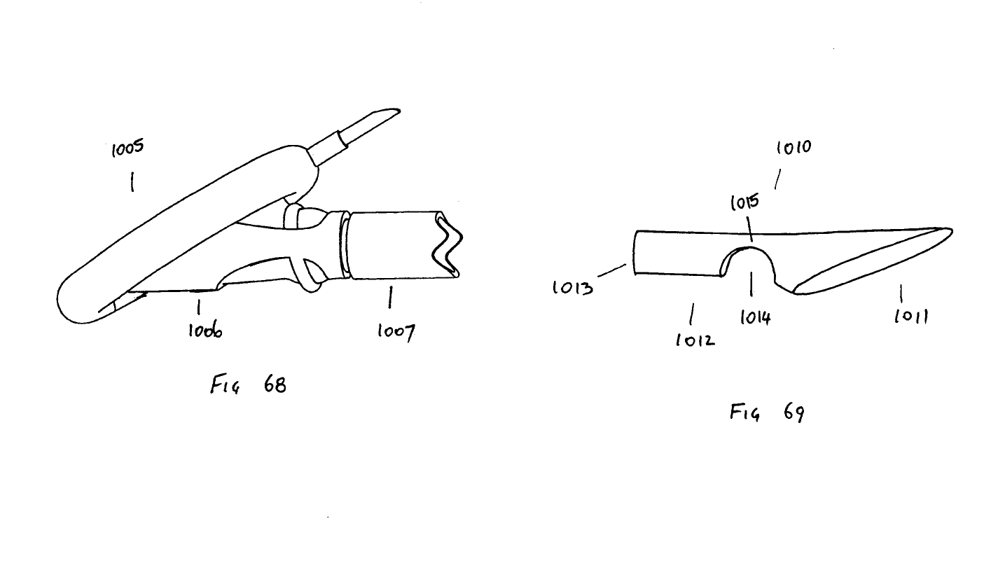

FIG. 69 shows a view of an alternative connector 1010 that may be used to connect a mask portion to an airway tube. The connector 1010 comprises a distal end 1011 that is shaped to fit into a chamber region of a mask portion. An airway extension 1012 extends in a proximal direction from the distal end 1011. The proximal end 1013 can be connected to an airway tube. An opening or space 1014 is defined in the connector 1010. A dorsal wall 1015 bounds a dorsal side of the opening or space. The ventral wall 1015 allows thrust to be transmitted in the along. The combination of the ventral wall 1015 and the opening or space 1014 allows flexing around the ventral wall 1015 and opening 1014. This region can function as a hinge joint (as can similar regions in the mask portion of other embodiments of the present invention).

FIG. 70 shows a side view of a mask portion 1100 for use in a laryngeal mask for establishing an artificial airway in a patient. The mask portion 1100 includes a backing plate 1102. The backing plate 1102 has an inflatable cuff 1104 located on a ventral side of the backing plate. The backing plate 1102 defines a chamber that, in use, has an opening that is positioned over the larynx of the patient. The inflatable cuff 1104 assists in making a seal around the larynx. Ventilation gases or anaesthetic gases are provided via an airway tube that is connected to the airway extension 1106. The ventilation gases or anaesthetic gases pass through the airway extension 116, into the chamber defined by the backing plate 1102 and thereafter into and through the larynx of the patient to thereby pass into the lungs of the patient.

FIG. 71 shows a cross sectional view of a conventional prior art laryngeal mask portion. As can be seen from FIG. 71, the backing plate 1102 defines a chamber 1103. The inflatable cuff 1104 is also shown in cross-section.

In order to manufacture the mask portion 1100 shown in FIG. 71, the backing plate 1102 and the airway extension 1106 are formed, typically by moulding, such as by injection moulding. The backing plate 1102 has a skirt extending downwardly from approximately the region showing by reference numeral 1108 in FIG. 71. An outwardly extending projection or lip 1110 is also formed on an outer surface of the backing plate 1102.

In order to form the cuff 1104, the skirt is folded such that the free end 1112 is placed in close proximity to the projection 1110. A line of glue 1114 is then applied to glue the free end of the skirt to the projection 1110 and outer wall of the backing plate 1102 to form the inflatable cuff 1104.

This method of manufacturing the inflatable cuff requires that a relatively large amount of glue be used. This heavy line of glue has the effect of decreasing the flexibility of the mask portion, thereby making the mask more difficult to insert. An alternative embodiment for making the inflatable cuff is described hereunder.

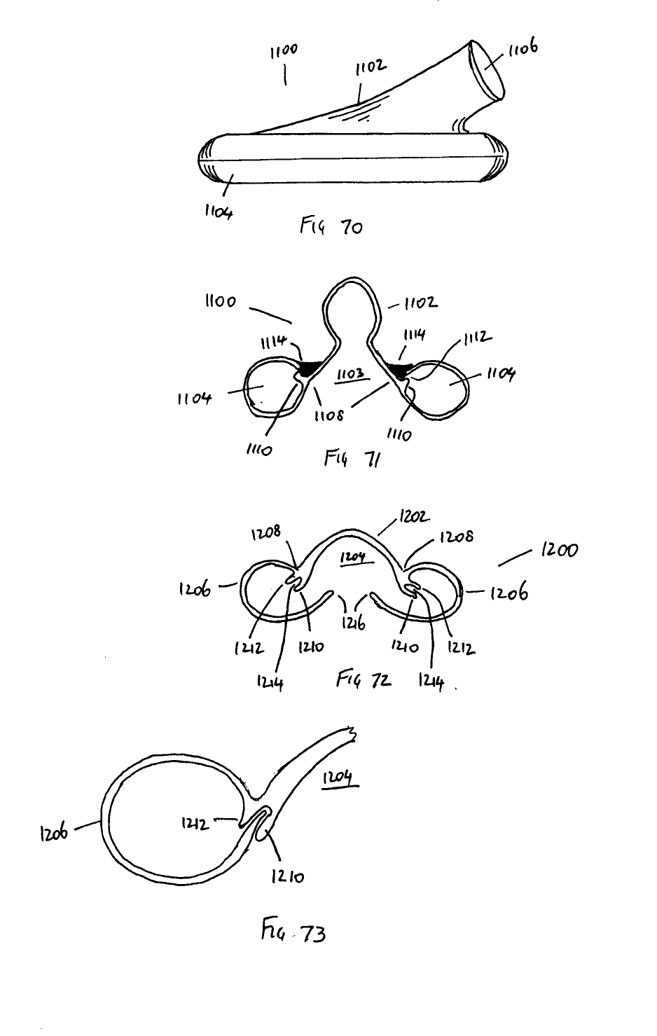

FIG. 72 shows a cross sectional view of a mask portion following formation, such as by moulding, of the mask portion, but before completion of the inflatable cuff. As can be seen from FIG. 72, the mask portion 1200 has a backing plate 1202 that defines a chamber 1204. The backing plate has a skirt 1206 extending therefrom. The skirt 1206 extends around the backing plate 120 such that the base of the skirt 1208 circumscribes a closed line. The backing plate 1202 also has two spaced lips 1210, 1212 located on a ventral side thereof. A space 1214 is defined between the two spaced lips 1210, 1212. In the embodiments shown in FIG. 72, the spaced lips 1210, 1212 are located ventrally of the base 1208 of the skirt 1206.

The skirt 1206 has a free end 1216. Advantageously, the circumferential length of the free end 1216 of the skirt 1206 is less then the circumferential length defined by the spaced lips 1210, 1212.

In order to complete the manufacture of the inflatable cuff, the free end 1216 of the skirt 1206 is inserted into the space 1214 defined between the two spaced lips 1210, 1212. This is shown in FIG. 73. If the circumferential length of the free end 1216 of the skirt 1206 is less then the circumferential length defined by the spaced lips 1210, 1212, it will be necessary to stretch the free end of the skirt 1206 in order to insert the free end 1216 of skirt 1206 into the space 1214. Therefore, the skirt will effectively “self tension” itself and this will assist in retaining the end 1216 of the skirt 1206 in the space 1214. Thus, assembly and manufacture of the inflatable cuff without requiring glue may be achieved. Of course, it may be sensible or prudent for the manufacturer to provide a small amount of glue in the space 1214 or between the end 1216 of the skirt and one or both of the lips 1210, 1212. However, it is likely that a significantly smaller amount of glue will be required than for the prior art embodiment shown in FIG. 71. Accordingly, the mask portion manufactured in accordance with FIGS. 72 and 73 will have greater flexibility than the mask portion shown in FIG. 71.

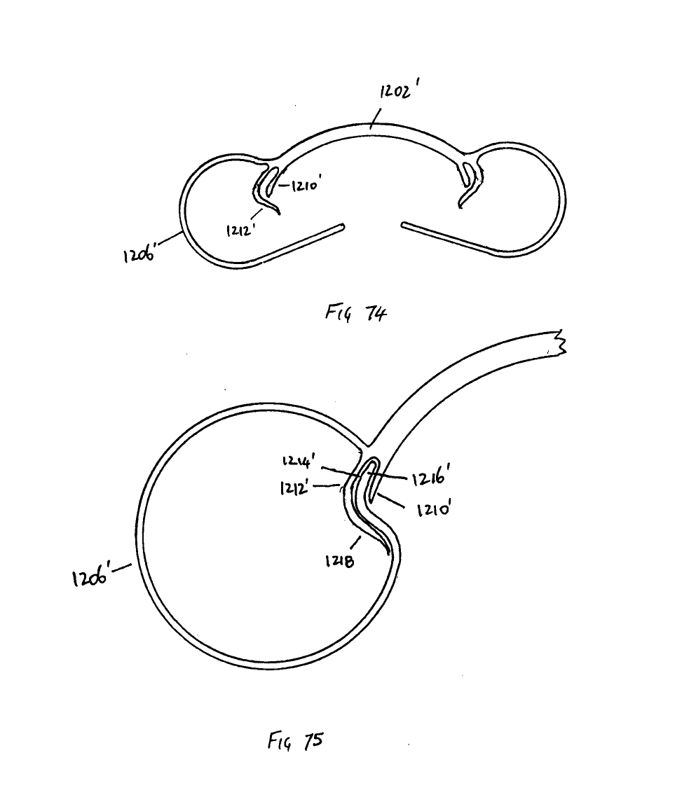

FIGS. 74 and 75 show another embodiment of the method described in relation to FIGS. 72 and 73. The features and methods to shown in FIGS. 74 and 75 are generally similar to those as shown and described in FIGS. 72 and 73. Therefore, like reference numerals will be used to denote similar features, but with the addition of a ′ in FIGS. 74 and 75. The embodiment shown in FIGS. 74 and 75 differs from the embodiment shown in FIGS. 72 and 73 in that lip 1212′ has an inward extension 1218 formed thereon. This provides further support for the skirt 1206′ when the free end 1216′ of the skirt is positioned in the space 1214′.

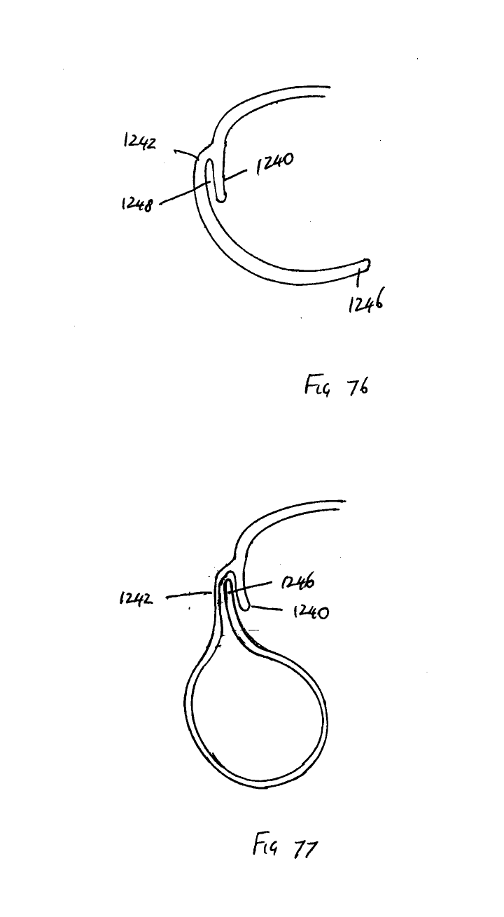

FIG. 76 shows a cross sectional view of an alternative embodiment of a mask portion following formation, such as by moulding, of the mask portion, but before completion of the inflatable cuff. Only the left side is shown in FIG. 76, with the right side being a mirror image of the left side. Unlike the embodiments shown in FIGS. 72 to 75, the embodiment shown in FIG. 76 utilises a single lip 1240 that is spaced from the base 1242 of the skirt 1244. In order to complete the inflatable cuff, the free end of the skirt 1246 is inserted into the space 1248 defined between the lip 1240 and the base 1242 of the skirt 1244, as shown in FIG. 77.

Those skilled in the art will appreciate that the present invention may be susceptible to variations and modifications other than those specifically described. It will be understood that the present invention encompasses all such variations and modifications that fall within its spirit and scope.

1. A laryngeal mask for establishing an artificial airway in a patient, comprising a mask portion having a resilient conformable peripheral portion shaped such that the mask forms a seal with the larynx when the mask is positioned in the laryngopharynx, and an airway tube connected to or formed with the mask for passing gas to the larynx when the mask is properly inserted into the laryngopharynx, the mask portion including at least one region of reduced wall thickness or enhanced flexibility, and at least one region of increased wall thickness (relative to the reduced wall thickness) or enhanced stiffness (relative to the region of enhanced flexibility), the at least one region of increased wall thickness or enhanced stiffness having a larger longitudinal extent then a longitudinal extent of the at least one region of reduced wall thickness or enhanced flexibility.

2. A laryngeal mask as claimed in claim 1 wherein the at least one region of increased wall thickness or enhanced stiffness is located adjacent to the at least one region of reduced wall thickness or enhanced flexibility.

3. A laryngeal mask as claimed in claim 1 or claim 2 wherein the at least one region of increased wall thickness or enhanced stiffness provides a line of material that extends past the at least one region of reduced wall thickness or enhanced flexibility and beyond either end of the at least one region of reduced wall thickness or enhanced flexibility such that the at least one region of increased wall thickness or enhanced stiffness provide a line of material that can support and transmit a thrusting force applied to the laryngeal mask during insertion thereof into a patient.

4. A laryngeal mask as claimed in any one of the preceding claims wherein the at least one region of reduced wall thickness comprises a ventral region or a dorsal region.

5. A laryngeal mask as claimed in any one of the preceding claims wherein the at least one region of reduced wall thickness comprises two regions of reduced wall thickness.

6. A laryngeal mask as claimed in claim 5 wherein one of the regions of reduced wall thickness is located on a ventral side of the mask portion and the other of the regions of reduced wall thickness is located on a dorsal side of the mask portion.

7. A laryngeal mask as claimed in any one of the preceding claims wherein the at least one region of increased wall thickness or enhanced stiffness in the mask portion of the mask is provided in a proximal end of the mask portion.

8. A laryngeal mask as claimed in claim 5 wherein the at least one region of increased wall thickness or enhanced stiffness is located close to a region where the mask is connected to the airway tube or, if the airway tube and mask portion are integrally formed with each other, close to the region where the mask portion merges into the airway tube.

9. A laryngeal mask for establishing an artificial airway in a patient, comprising a mask portion having a resilient conformable peripheral portion shaped such that the mask forms a seal with the larynx when the mask is positioned in the laryngopharynx, and an airway tube connected to or formed with the mask for passing gas to the larynx when the mask is properly inserted into the laryngopharynx, the airway tube including at least one region of enhanced flexibility located at or near a distal end of the airway tube, the airway tube including a continuous region having increased stiffness (relative to the region of enhanced flexibility), the continuous region of increased stiffness extending from the distal end of the airway tube to a proximal end of the airway tube to thereby provide a continuous line of enhanced stiffness to transmit a pushing force therealong, the one or more regions of enhanced flexibility comprising one or more regions having an essentially identical wall thickness to the rest of the airway tube, but having a configuration that enhances flexibility.

10. A laryngeal mask as claimed in claim 9 wherein the one or more regions of enhanced flexibility comprise one or more regions having a similar wall thickness to the rest of the airway tube, but having a configuration that enhances flexibility.

11. A laryngeal mask as claimed in claim 10 wherein the one or more regions of enhanced flexibility comprises a corrugated region or a bellows type region.

12. A laryngeal mask as claimed in any one of claims 9 to 11 wherein the at least one region of enhanced flexibility located at or near a distal end of the airway tube comprises a ventral region or a dorsal region.

13. A laryngeal mask as claimed in any one of claims 9 to 12 wherein the at least one region of enhanced flexibility comprises two regions of enhanced flexibility.

14. A laryngeal mask as claimed in claim 13 wherein one of the regions of enhanced flexibility is located on a ventral side of the airway tube and the other of the regions of enhanced flexibility is located on a dorsal side of the airway tube.

15. A laryngeal mask as claimed in any one of claims 9 to 14 wherein the at least one region of enhanced flexibility extends around part of a circumference of the airway tube.

16. A laryngeal mask as claimed in any one of claims 9 to 15 wherein the at least one region of enhanced flexibility extends along a longitudinal direction of the airway tube.

17. A laryngeal mask as claimed in claim 16 wherein the at least one region of enhanced flexibility suitably extends only along a portion of the longitudinal extent of the airway tube.

18. A laryngeal mask for establishing an artificial airway in a patient, comprising a mask portion having a resilient conformable peripheral portion shaped such that the mask forms a seal with the larynx when the mask is positioned in the laryngopharynx, and an airway tube connected to or formed with the mask for passing gas to the larynx when the mask is properly inserted into the laryngopharynx, the mask portion including a base plate that, in use, is positioned dorsally of the resilient conformable peripheral portion, the base plate being formed with the airway tube or being connected to the airway tube, wherein the base plate includes at least one region of reduced wall thickness or enhanced flexibility, the base plate also including at least one region of thicker wall thickness or increased stiffness (relative to the at least one region of reduced wall thickness or enhanced flexibility, respectively), the region of thicker wall thickness or increased stiffness extending from a distal part of the base plate to a part of the base plate that joins with or merges into the airway tube.

19. A laryngeal mask as claimed in claim 18 wherein the at least one region of reduced wall thickness or enhanced flexibility in the base plate is located at a dorsal part of the base plate with the at least one region of thicker wall thickness or increased stiffness being located to one side of the at least one region of reduced wall thickness or enhanced flexibility.

20. A laryngeal mask as claimed in claim 19 wherein the base plate has regions of thicker wall thickness or increased stiffness located on either side of a dorsal region of reduced wall thickness or enhanced flexibility.

21. A laryngeal mask as claimed in any one of claims 18 to 20 wherein the at least one region of reduced wall thickness or enhanced flexibility in the base plate is located at a ventral part of the base plate with the at least one region of thicker wall thickness or increased stiffness being located to one side of the at least one region of reduced wall thickness or enhanced flexibility.

22. A laryngeal mask as claimed in claim 21 wherein the base plate has regions of thicker wall thickness or increased stiffness located on either side of the ventral region of reduced wall thickness or enhanced flexibility.

23. A laryngeal mask as claimed in claim 18 wherein one region of reduced wall thickness or enhanced flexibility in the base plate is located in a dorsal part of the base plate and another region of reduced wall thickness or enhanced flexibility in the base plate is located in a ventral part of the base plate, with the at least one region of thicker wall thickness or increased stiffness comprising two regions located circumferentially between the dorsal and ventral regions of reduced wall thickness or enhanced flexibility.

24. A laryngeal mask as claimed in any one of the preceding claims wherein the resilient conformable peripheral portion shaped such that the mask forms a seal with the larynx comprises an inflatable cuff or a non-inflatable portion or a soft, non-inflatable cuff that fits snugly onto the perilaryngeal framework, mirroring the shape of the epiglottis, aryepiglottic folds, piriform fossae, peri-thyroid, peri-cricoid, posterior cartilages and spaces.

25. A laryngeal mask as claimed in any one of the preceding claims further comprising one or more cavities or tubes that, in use, have an opening that extends into the oesophagus of the patient.

26. A laryngeal mask as claimed in claim 25 wherein the distal portion of the laryngeal mask is provided with an extension that opens the upper oesophagus.

27. A laryngeal mask as claimed in claim 26 wherein the extension extends distally beyond the distal extent of the openings of the one or more cavities or tubes.

28. A laryngeal mask as claimed in any one of the preceding claims wherein the laryngeal mask is provided with deformation means located on the mask, wherein the application of force to the deformation means causes elastic deformation of the device, thereby facilitating insertion of the device into the patient.

29. A laryngeal mask for establishing an artificial airway in a patient, comprising a mask portion having a resilient conformable peripheral portion shaped such that the mask forms a seal with the larynx when the mask is positioned in the laryngopharynx, and an airway tube connected to or formed with the mask for passing gas to the larynx when the mask is properly inserted into the laryngopharynx, characterised in that the laryngeal mask includes a joint adapted to permit relative rotation between the airway tube and the mask portion.

30. A laryngeal mask as claimed in claim 29 wherein the joint comprises a swivel joint or a hinge joint or a ball and socket joint or a joint that allows for rotation about at least two different axes.

31. A laryngeal mask as claimed in claim 29 or claim 30 wherein the joint is arranged such that a thrusting force applied to the airway tube is transmitted through the joint to the mask portion of the device.

32. A laryngeal mask as claimed in any one of claims 29 to 31 wherein the joint may allows flexing and extension during insertion of the mask to facilitate the mask following the airway of the patient during the insertion procedure.

33. A laryngeal mask as claimed in any one of claims 29 to 32 wherein the joint has an external flexion to assist in maintaining the desired flex of the mask portion to help the mask portion slide behind the larynx.

34. A laryngeal mask as claimed in any one of claims 29 to 33 wherein the joint is arranged such that restricted relative rotation between the mask portion and the airway tube is allowed.

35. A laryngeal mask as claimed in claim 34 wherein the joint is provided with a stop member to cause restricted relative rotation.

36. A laryngeal mask as claimed in any one of claims 29 to 35 wherein the mask portion has a connector region, the connector region forming part of the joint or receiving part of the joint, the connector region including a region of reduced sidewall thickness or a region of enhanced flexibility or a region where the sidewall is not present (either through the sidewall not being formed in that region during manufacture or by removal of the sidewall).

37. A laryngeal mask as claimed in claim 36 wherein the connector region is positioned at or near a proximal end of the mask portion.

38. A laryngeal mask for establishing an artificial airway in a patient, comprising a mask portion having a resilient conformable peripheral portion shaped such that the mask forms a seal with the larynx when the mask is positioned in the laryngopharynx, and an airway tube connected to or formed with the mask for passing gas to the larynx when the mask is properly inserted into the laryngopharynx, wherein the mask portion includes a dorsal surface, characterised in that a line extending from a proximal part to a distal part of a dorsal-most part of the dorsal surface extends generally parallel to a longitudinal axis of a distal most part of the airway tube, or the line extending from a proximal part to a distal part of a dorsal-most part of the dorsal surface extends towards a ventral region in a direction from the proximal to distal part of that line.

39. A laryngeal mask for establishing an artificial airway in a patient, comprising a mask portion having a resilient conformable peripheral portion shaped such that the mask forms a seal with the larynx when the mask is positioned in the laryngopharynx, and an airway tube connected to or formed with the mask for passing gas to the larynx when the mask is properly inserted into the laryngopharynx, wherein the mask portion includes a base plate, the base plate having a dorsal surface, characterised in that a line extending from a proximal part to a distal part of a dorsal-most part of the dorsal surface extends generally parallel to a longitudinal axis of a distal-most part of the airway tube or the line extends at an angle to the longitudinal axis of a distal most part of the airway tube such that the line extends distally and ventrally from a proximal end of the line relative to the longitudinal axis of a distal most part of the airway tube.

40. A method for manufacturing a laryngeal mask, the laryngeal mask including a mask portion defining a chamber, and an inflatable cuff extending around the chamber, the chamber being in fluid communication with an airway tube such that gases supplied from the airway tube pass into the chamber and thereafter into the larynx of a patient when the mask is in use, the method comprising the steps of:
a) forming the mask portion such that a skirt extends from the mask portion, the mask portion having at least one lip spaced from a base of the skirt or two spaced lips;
b) inserting a free end of the skirt into a space defined between the at least one spaced lip and a base of the skirt or between the two spaced lips; and
c) retaining the free end of the skirt between the at least one spaced lip and the base of the skirt or between two spaced lips such that the skirt defines a substantially closed outer surface and can function as an inflatable cuff.

41. A method as claimed in claim 40 wherein a circumferential length of the free end of the skirt is less then a circumferential length of the space between the at least one spaced lip and the base of the skirt or between two spaced lips.

42. A method as claimed in claim 41 wherein the circumference of the free end of the skirt is stretched to fit the skirt into the space.

43. A method as claimed in any one of claims 40 to 42 wherein the free end of the skirt is glued in position between the base of the skirt and the lip or between the two spaced lips to retain the free end of the skirt, or ultrasonic welding is used to retain the free end of the skirt in position between the base of the skirt and the lip or between the two spaced lips to retain the free end of the skirt, or a suitable double sided adhesive tape is used to retain the free end of the skirt in position between the base of the skirt and the lip or between the two spaced lips to retain the free end of the skirt, or a friction fit is used to retain the free end of the skirt in position between the base of the skirt and the lip or between the two spaced lips or the at least one lip or the two spaced lips comprise resilient lips and the cuff may be formed by inserting the free end of the skirt between the between the base of the skirt and the lip or between the two spaced lips with the resilience of the lip or lips holding the free end of the skirt in position.

44. A method as claimed in any one of claims 40 to claim 43 wherein the mask portion is formed by a moulding process.

45. A method as claimed in claim 44 wherein the moulding process also forms the skirt and the at least one lip or the two spaced lips.

46. A method as claimed in claim 45 wherein the chamber, the skirt and the at least one lip or the two spaced lips are formed in a single moulding process.

47. A method as claimed in any one of claims 40 to 46 wherein the at least one lip or the two spaced lips circumscribe a shape that forms part of the shape of the cuff in the completed mask.

48. A method as claimed in any one of claims 44 to 47 wherein when the mask portion is removed from the mould, the skirt extends away from the mask portion and the skirt extends around the mask portion, with a free end of the skirt being located away from the mask portion.

49. A method as claimed in any one of claims 40 to 48 wherein step (c) comprises inserting the free end of the skirt into the space between the at least one lip and the base of the skirt or between the two lips and fixing the free end of the skirt in place such that the skirt defines the inflatable cuff.

50. A method as claimed in any one of claims 40 to 49 wherein the skirt includes an inlet that defines a passage for receiving inflation air from an inflation tube so that the inflatable cover can be selectively inflated and deflated.

51. A method as claimed in claim 50 wherein the inflation tube is permanently connected to the inlet of the cuff.

52. A laryngeal mask including a mask portion defining a chamber, and an inflatable cuff extending around the chamber, the chamber being in fluid communication with an airway tube such that gases supplied from the airway tube pass into the chamber and thereafter into the larynx of a patient when the mask is in use, wherein the inflatable cuff is formed from a skirt extending from the mask portion and having an end of the skirt being positioned between and retained between at least one lip and a base of the skirt or between two spaced lips.

53. A laryngeal mask as claimed in claim 50 wherein the at least one lip or the two spaced lips extend from a ventral part of the chamber.
